(19) United States
(12) Patent Application Publication
(10) Pub. No.: US 2026/0132456 A1
(43) Pub. Date: May 14, 2026

(54) Methods for Extraction and Sequencing of Nucleic Acid

(71) Applicants: Earlham Enterprises Ltd, Norwich (GB); The Natural History Museum Trading Company Limited, London (GB)

(72) Inventors: Richard Leggett, Norwich (GB); Matthew Clark, London (GB); Raju Misra, London (GB); Darren Chooneea, London (GB); Michael Giolai, London (GB); Pia Aanstad, London (GB); Piotr Cuber, London (GB); Darren Heavens, Norwich (GB)

(21) Appl. No.: 18/998,506

(22) PCT Filed: Jul. 27, 2023
(86) PCT No.: PCT/GB2023/051992

§ 371 Date: Jan. 26, 2025

(30) Foreign Application Priority Data
Jul. 27, 2022 (GB) 2210963.1

(51) Int. Cl. C12Q 1/6874; C12N 15/10; G01N 1/04; G01N 1/22

(52) U.S. Cl. CPC C12Q 1/6874; C12N 15/1013; G01N 1/04; G01N 1/2273

(57) ABSTRACT

The present invention relates to methods for extracting nucleic acids from a sample comprising one or more cells. The methods comprise steps of lysing and mechanically disrupting cells in order to provide optimum parallel extraction of both DNA and RNA from samples. The invention also relates to methods of identifying pathogens and organism profiling of samples.

### FIELD

The present invention relates to methods for the extraction of nucleic acids from environmental samples with low nucleic acid abundance.

### BACKGROUND

Nucleic acid sequencing is a fundamental part of modern biology and has a wide range of applications. For example, sequencing can be used to identify and classify species such as pathogens with samples obtained from the environment. A key step required for successful analysis of nucleic acid within a sample is the extraction of the nucleic acid from the cells, wherein the nucleic acid is selectively retained whilst other components of the cells and sample are removed. Effective extraction requires that the cells are effectively lysed and then the released nucleic acid recovered. There are three primary methods which are used to achieved lysis of cells in order to extract nucleic acid these are mechanical, chemical and enzymatic. The type of cells in the sample and the downstream processing steps may influence which approach is most suitable.

Samples obtained from the environment may comprise a variety of different cells from various organisms including those with DNA or RNA encoded genomes, or RNA: DNA hybrid genomes. For example, environmental sample may comprise both viruses and bacteria along with other pathogens or non-pathogenic species. The environmental sample may also comprise larger organisms such as plants and insects. As such, a parallel method for the extraction of DNA and RNA would be advantageous in order to successfully identify all of the microorganisms present. However, to date, the amplification and sequencing of environmental RNA has been hampered by issues with sensitivity, range of detection, and time required. Most studies have relied on targeted qRT-PCRs using specific primers for a limited range of known viruses, an approach which precludes the unbiased detection of novel or unsuspected species. Although some studies have used non-targeted RNA sequencing, this approach requires very large volumes of starting material (air collection for several days/weeks, or large volumes of wastewater). An additional problem in non-targeted environmental RNA sequencing approaches is that some current commercial viral RNA extraction kits include carrier RNA, which significantly decreases the efficiency of metatranscriptomics sequencing, as a significant percentage of the sequence reads will be this carrier RNA. Most RNA sequencing methods convert the RNA to cDNA for library and platform compatibility, but any contaminating gDNA will also be sequenced. Therefore, there is a need for improved nucleic acid extraction methods.

### SUMMARY OF THE INVENTION

There is evidence that complete lysis of bacterial cell walls is critical for optimum yield of nucleic acid. Lysis protocols include procedures that lead to physical and or enzymatic disruption of the cell wall or cell membrane. It has been observed that extended lysis time and mechanical disruption can enhance nucleic acid yield. However, extended lysis time can also shear the genomic DNA into smaller fragments, which may not allow accurate sequencing and identification of the organisms present in a sample. In general, cells are lysed to release the nucleic acids and the remaining proteins are discarded.

The present inventors have developed an optimal method for the parallel extraction of both DNA and RNA from samples with a low abundance of nucleic acid or biomass. In particular this method is useful in the extraction of nucleic acids from environmental samples such as air samples and may be used in methods of monitoring the organisms that are present in the air. The method combines the use of a lysis buffer comprising a chaotropic agent, such as guanidine, and bead beating. The present method advantageously allows excellent extraction of nucleic acid, whilst also minimising fragmentation of the nucleic acid. As shown, herein the extraction method allows longer nucleic acid molecules to be obtained which can be subsequently amplified and sequenced. As such, this extraction method can be combined with steps of amplifying and sequencing the nucleic acid. The present method provides long reads of nucleic acids. These long reads provide a rich data set that allow the sequence reads to be assigned to a specific organism, the long reads can also be uniquely assigned at high confidence to a specific species or even strain. Further, wherein the sample contains a unique organism or pathogen the long reads are advantageous for genome assembly.

An aspect of the invention relates to a method for extracting a nucleic acid from a sample comprising one or more cells comprising nucleic acid, the method comprising:
providing a sample comprising one or more cells;
lysing the one or more cells by contacting the sample with a solution comprising a chaotropic agent; and
mechanically disrupting the sample comprising one or more cells;
contacting the sample with magnetic particles;
wherein the method comprises contacting the sample with a non-RNA carrier molecule.

The chaotropic agent may be a chaotropic salt. The chaotropic agent may be selected from phenol, ethanol, guanidinium, urea, iodide and lithium perchlorate. In an embodiment the chaotropic agent is guanidinium. In an embodiment the non-RNA carrier is selected from LPA or glycogen. In an embodiment the solution comprising a chaotropic agent is used at a volume between 50 μl to 250 μl. In an embodiment the sample is contacted with between 100 mg to 350 mg of the magnetic particles. In an embodiment the method further comprises a step of amplifying the nucleic acid to produce an amplified nucleic acid sample. In an embodiment the amplification step comprises amplifying DNA via whole genome amplification, to produce a sample of amplified DNA. In an embodiment the amplification step comprises amplifying DNA via Multiple strand Displacement Amplification In an embodiment the method comprises a step of debranching the amplified DNA, comprising contacting the amplified DNA with an endonuclease selected from SI, T7. In an embodiment the amplification step comprises amplifying RNA via a method selected from RT-PCR, isothermal amplification or rolling circle amplification to produce a sample of amplified RNA. In an embodiment the amplification step comprises a step of polyadenylating RNA. In such embodiments, the amplification step may further comprise reverse transcription using an oligonucleotide deoxythymidine homopolymer primer. In an embodiment the amplification step comprises copying the RNA with a reverse transcriptase preferably Superscript IV. In an embodiment the amplification step comprises amplifying the RNA with primers that attach a CLICK chemistry active group to the amplified cDNA. In an embodiment the CLICK group is selected from a dibenzocyclooctyne group, or an azide group. In an embodiment the method further comprises a step of sequencing the nucleic acid. In an embodiment a bioinformatics method may be used to determine the species of the cells present in the sample. In an embodiment the sequencing comprises whole genome sequencing, whole exome sequencing, targeted sequencing or metagenomic sequencing. In an embodiment the sample is a sample obtained from the environment. In an embodiment the sample is an air, water, soil sample. In an embodiment the air sample is collected via an air sample filter. In an embodiment the air sample filter is Coriolis micro, FLIR IBAC 2, ACD-200 Bobcat a. In an embodiment the sample is filtered prior to performing the extraction of nucleic acid. In an embodiment the step of sequencing comprises sequencing nucleic acid molecules that are 0.5 kb to 30 kb in length.

An aspect of the invention relates to a method for identifying pathogens and/or allergens present in an environmental sample, the method comprising
obtaining a sample from the environment comprising one or more cells;
extracting nucleic acid from said sample by lysing the one or more cells by contacting the sample with a solution comprising a chaotropic agent; mechanically disrupting the sample comprising one or more cells; contacting the sample with magnetic particles, contacting the sample with a non-RNA carrier molecule;
amplifying the nucleic acid; and
sequencing the nucleic acid.

An aspect of the invention relates to a method for organism profiling of an environmental sample, the method comprising
obtaining a sample from the environment comprising one or more cells;
extracting nucleic acid from said sample by lysing the one or more cells by contacting the sample with a solution comprising a chaotropic agent; mechanically disrupting the sample comprising one or more cells; contacting the sample with magnetic particles; contacting the sample with a non-RNA carrier molecule;
amplifying the nucleic acid; and
sequencing the nucleic acid and identifying the microorganisms present in the environmental sample.

An aspect of the present invention relates to a kit for the extraction of nucleic acid, comprising a solution comprising a chaotropic agent (for example guanidinium), magnetic particles, a non-RNA carrier and optionally instructions for use.

### FIGURES

FIG. 1: Flowchart showing the steps of extracting and sequencing nucleic acid from samples comprising RNA viruses and DNA microbes (viruses, bacteria and fungi);

FIG. 2: Analysis of DNA and RNA fragment size recovery;

FIG. 3: A) D5000 screen tape analysis shows different size selection with the different beads used in this experiment. Shown are 2 technical replicates for each of the bead protocols from the first experiment only. B) The plot shows the mean percentage of DNA recovered from 3 technical replicates, with 3 independent experiments for each protocol. Error bars indicate standard deviation;

FIG. 4: DNA quality extracted with different combinations of the extraction beads and lysis buffers visualized on TapeStation;

FIG. 5: DNA yield and quality extracted with different combinations of the extraction beads and lysis buffers visualized on TapeStation;

FIG. 6: Tapestation analysis of extracted RNA (left) and PCR products (right) of an air sample amplified using the RNA pipeline. The strong RNA band is likely to be ICV RNA that was used to spike the air sample;

FIG. 7: Quantification and analysis of mcSCRBseq yields given different RNA inputs, RT incubation times and PCR extension times. The graphs show the total yield from n=1 experiment, with the x axis representing the RT incubation time, and PCR extension times as follows: 30 sec in green, 45 sec in blue, 1 min in red, and 2 min in orange;

FIG. 8: Electropherograms showing on-bead versus off-bead WGA;

FIG. 9: Timing improvements observed in different pipelines;

FIG. 10: S1 nuclease digest sequencing performance: Both reactions sequenced for a similar amount of time, however, in the WGA unclean+S1 reaction higher Adapter content and a faster loss of pores i.e. “no pore from scan” was observed. S1 nuclease digest sequencing performance: higher pore saturation rates in the WGA unclean+S1 reaction was observed with a steeper decrease of available single pores over time; and

FIG. 11: Mux scans of WGA alone, WGA×S1 and WGA×T7.

### DETAILED DESCRIPTION OF THE INVENTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

The present inventors have developed a method in which RNA extraction is performed simultaneously with DNA using a bead-based protocol, thus streamlining the process and allowing the monitoring of both RNA and DNA from environmental samples. It has been demonstrated herein that RNA can be extracted from low volume air samples without the need for addition of carrier RNA. The inventors have also modified existing single cell sequencing protocols for the amplification of environmental RNA samples and show that minimally biased metatranscriptomic sequencing can be performed with as little as 5-10 pg of starting RNA. Further optimisations have enhanced the time efficiency of the amplification protocol. Using these methods, it has been demonstrated that low (pg) amounts of environmental RNA can be extracted, and used for specific and unbiased amplification followed by metatranscriptomic sequencing.

### Nucleic Acid Extraction

The present inventors have aimed to identify a robust and efficient method for extracting nucleic acid from challenging cell types, in order to profile microbial and eukaryote diversity in samples. After cell collection, cell lysis and nucleic acid extraction are the first critical steps for comprehensive profiling of microbial diversity. Described herein is a method for the parallel extraction of DNA and RNA from cells. The present inventors have shown herein that the method is suitable to extract both DNA and RNA from cells which allows the genetic material from organisms such as viruses, bacteria, fungi and other eukaryotes to be obtained in parallel achieving comprehensive profiling of pathogens present in environmental samples. This method is compatible with further steps such as amplification and sequencing to allow identification of the species present in the sample.

An aspect of the invention relates to a method for extracting a nucleic acid from a sample comprising one or more cells comprising nucleic acid, the method comprising:
providing a sample comprising one or more cells;
lysing the one or more cells by contacting the sample with a solution comprising a chaotropic agent;
mechanically disrupting the sample comprising one or more cells; and
contacting the sample with magnetic particles;
wherein the method comprises contacting the sample with a non-RNA carrier molecule.

The term “nucleic acid” as used herein refers to single- or double-stranded DNA and RNA. The term “DNA” includes but is not limited to genomic DNA and cDNA. The term “RNA” includes but is not limited to mRNA, RNA, RNAi molecules including siRNA, microRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylcytosine, hydroxymethylcytosine, formylcytosine, carboxylcytosine, oxoguanine, oxoadenine, methyladenosine, and/or thiouridine, although without limitation thereto.

The sample is a biological sample i.e., a sample comprising or suspected of comprising biological material. The biological sample may be a fluid containing cells and/or nucleic acids. Typical samples which can be used in the methods of the invention include bodily fluids such as blood, which can be anticoagulated blood as is commonly found in collected blood specimens, plasma, serum, urine, semen, saliva, cell cultures, tissue extracts and the like. Other types of samples include solvents, seawater, wastewater or sewage, industrial water samples, food samples and environmental samples such as air, soil or water, plant materials, eukaryotes, bacteria, plasmids and viruses, fungi, and cells originated from prokaryotes. The present method is particularly useful for extracting nucleic acid from samples comprising a low abundance of nucleic acid, for example samples obtained from the air.

The sample is contacted with a solution comprising a chaotropic agent. The chaotropic agent may be a chaotropic salt. The chaotropic agent may be selected from phenol, ethanol, guanidinium, urea, iodide and lithium perchlorate. In an embodiment the sample is contacted with a solution comprising guanidinium in order to lyse the cells, preferably the solution comprises guanidine thiocyanate and/or guanidine hydrochloride. Guanidine thiocyanate and guanidine hydrochloride are potent chaotropic agents which interfere with the hydrogen bond network in aqueous solutions, and have a destabilizing effect on macromolecules, in particular proteins. Advantageously, chaotropic agents such as guanidine thiocyanate and/or guanidine hydrochloride can lyse virus particles to extract nucleic acids and denature RNAse and DNAse enzymes that may otherwise damage the extract.

The step of mechanically disrupting the sample comprising one or more cells acts to mechanically lyse the cells. The mechanical lysis may be achieved by a process called “bead beating”, wherein moderate to high-speed movement is applied to the sample containing particles or “beads” causing collisions between the beads and the samples. The step of mechanical disrupting the sample may comprise contacting the sample with beads or particles and homogenising said sample. In an embodiment the beads or particles may be steel, garnet, zirconium carbide, silicon carbide, boron, or another hard material on the Mohs scale. Various devices are known in the art for performing bead beating. Suitable bead beaters include MP Biomedicals SuperFastPrep-2, Qiagen tissue lyser, Geno/Grinder, Omnilyse. In an embodiment the mechanical lysis is performed using Omnilyse—a small low powered bead beater. In embodiments the mechanical disruption of the sample is performed for between 1 to 20 minutes, 1 to 18 minutes, 1 to 16 minutes, 1 to 14 minutes, 1 to 12 minutes, 1 to 10 minutes, 1 to 8 minutes, 1 to 6 minutes. In an embodiment the mechanical disruption of the sample is performed for approximately 5 minutes e.g., 1 to 10 minutes, 2 to 8 minutes, 3 to 6 minutes.

The present method may use a combination of both chemical and mechanical lysis of cells to ensure that all cells within the sample are effectively lysed. This is particularly advantageous for samples comprising various cell types for example bacteria (including gram-negative and gram-positive), viruses, fungi. It is important to not only achieve lysis of all classes of biological material in the sample microbiome but also to balance that against the physical damage that bead beating or similar methods cause to RNA/DNA molecules, especially long genomic DNA molecules (which can be millions of bases long), as longer DNA fragments 1) have better yields in WGA 2) longer DNA sequences, once sequenced, can be better assigned to specific species or strains of microbe, which leads to lower false positive assignments to pathogens that share sequences with other (possibly related) non-pathogenic species.

The methods of the invention comprise a step of contacting the sample with magnetic particles. This step allows purification of the extracted nucleic acid for further processing. The magnetic particles may be magnetic or paramagnetic. The magnetic particles are used for purification of the nucleic acids, by binding the nucleic acids. Therefore, the method may comprise contacting the sample with magnetic particles to purify the nucleic acid. The paramagnetic or magnetic particles used in the methods of the present invention have a small size in diameter. The magnetic particles may be superparamagnetic particles which do not have magnetic interactions or aggregations without external magnetic field, so they can be well dispersed in solution and sufficiently adsorb the targets. Magnetic particles may be divided into three categories: metal oxides, pure metals and magnetic alloys. Co, Fe and Ni based magnetic particles are commonly used in biomedical applications. In one embodiment the magnetic particles are hydrophilic. In one embodiment carboxylate magnetic particles are used. Preferably magnetic particles comprising magnetite Fe₃O₄ or maghemite γ-Fe₂O₃ may be used for nucleic acid extraction as they show good biocompatibility, stability and fast separation under the external magnetic fields. In a preferred embodiment the magnetic particles are Sera-Mag Carboxylate-Modified Magnetic Particles. By using carboxylate-based magnetic particles non-specific binding to the particles can be reduced. The magnetic particles used in the present method allow selective precipitation of the nucleic acid onto the particles in order to effect purification. In an embodiment the sample may be contacted with the magnetic particles in the presence of polyethylene glycol (PEG). In an embodiment the PEG is PEG 8000. The PEG may be present at a concentration of 5 to 50% w/v, 5 to 40% w/v, 5 to 30% w/v, preferably approximately 20% w/v e.g., 5 to 25% w/v. In an embodiment the PEG may be of various lengths for example PEG 200, PEG 300, PEG 400, PEG 500, PEG 600, PEG 700, PEG 800, PEG 900, PEG 1000, PEG 2000, PEG 3000, PEG 4000, PEG 5000, PEG 8000, PEG 10 000. PEG 12 000, PEG 20 000. PEG derivative may also be used, PEG derivatives may include PEG ethers (e.g. laureths, ceteths, ceteareths, oleths, and PEG ethers of glyceryl cocoates), PEG fatty acids (e.g. PEG laurates, dilaurates, stearates, and distearates), PEG castor oils, PEG amine ethers (PEG cocamines), PEG propylene glycols, and other derivates (e.g., PEG soy sterols and PEG beeswax). In an embodiment the sample may be contacted with the magnetic particles in the presence of sodium chloride (NaCl). The NaCl may be present at a concentration between 1 to 5M, 1 to 4M, 1 to 3M, preferably approximately 2.5M e.g., 1.5 to 3M, 1.8 to 2.8M. In an embodiment the sample may be contacted with the magnetic particles at a temperature between 5 to 25° C., 6 to 24° C., 7 to 23° C. In one embodiment the sample may be contacted with the magnetic particles at a temperature between approximately 10 to 22° C.

The magnetic particles may have a size less than about 50 μm and more preferably less than about 10 μm. Small particles are more readily dispersed in solution and have higher surface/volume ratios. Larger particles and beads can also be useful in methods where gravitational settling or centrifugation are employed. Mixtures of two or more different sized particles may be advantageous in some embodiments. In an embodiment the magnetic particles are not silica based.

In an embodiment the step of contacting the sample with magnetic particles is performed subsequently to the step of mechanically disrupting the sample.

In the present method the sample is contacted with a non-RNA carrier molecule. The present inventors have shown herein that use of a non-RNA carrier improves the yield of nucleic acid, particularly RNA that can be extracted from the sample. Using a non-RNA carrier also has further advantages that the carrier molecule will not interfere with any downstream processing steps such as sequencing of the extracted nucleic acid. In contrast RNA carrier molecules which are commonly used to enhance extraction can negatively impact sequencing processes that may be used. In an embodiment the non-RNA carrier is selected from synthetic polymer, DNA, synthetic nucleic acid, or a polysaccharide or a combination thereof. In a preferred embodiment the non-RNA carrier is selected from synthetic polymer, DNA, synthetic nucleic acid, or a combination thereof. Suitable synthetic polymers include but are not limited to LPA (linear polyacrylamide). Suitable polysaccharides include but are not limited to glycogen. In an embodiment the non-RNA carrier is not glycogen. Suitable synthetic nucleic acids, include but are not limited to XNAs (xeno-nucleic acids), LNAs (locked-nucleic acids), PNAs (peptide nucleic acid). “Synthetic nucleic acid” refers to a non-naturally occurring nucleic acid which generally comprise a different sugar backbone than DNA or RNA. Such synthetic nucleic acids differ in some respect from nucleic acids that occur in nature without human intervention, whether by sequence, chemical composition, and/or functional properties. The term “locked nucleic acid” as used herein refers to a nucleic acid comprising modified RNA monomers. These Locked nucleic acids comprise a methylene bridge bond linking the 2′ oxygen to the 4′ carbon of the RNA pentose ring. The term “peptide nucleic acid” as used herein refers to a nucleic acid comprising synthetic mimics of DNA in which the deoxyribose phosphate backbone is replaced by a pseudo-peptide polymer to which the nucleobases are linked.

In order to enhance the yield of nucleic acid extracted from the sample a number of additional steps may be performed including using a filtration device to concentrate biological material prior to extraction. Suitable filter materials include, but are not limited to, PTFE (polytetrafluoroethylene), PES (polyethersulfone), cellulose acetate, SFCA (surfactant-free cellulose acetate), regenerated cellulose, nylon, polypropylene and/or a combination thereof. In an embodiment the filtration device comprises a filter material selected from PVDF (polyvinylidene fluoride) PTFE (polytetrafluoroethylene), PES (polyethersulfone), cellulose acetate, SFCA (surfactant-free cellulose acetate), regenerated cellulose, nylon, polypropylene and/or a combination thereof.

Further to enhance yield of nucleic acid lower volumes of reagents may be used. For example, the solution comprising a chaotropic agent may be used at a volume between 50 μl to 250 μl.

In an embodiment the sample is contacted with between 100 mg to 350 mg of the magnetic particles.

Once the extracted nucleic acid has been precipitated onto the magnetic particles in some embodiments the methods of the invention may comprise a step of eluting the nucleic acid from the magnetic particles. The step of elution may comprise eluting the nucleic acid using water. The step of elution may comprise eluting the nucleic acid using a buffer with a pH between pH 5.0 to 10.0, for example between pH 5.5 to 9.5, pH 6.0 to 9.0, pH 6.5 to 8.5, pH 7.0 to 8.0. In some embodiments a lower pH range may be beneficial for example a lower pH may be more effective at eluting RNA molecules. In embodiments where a low pH is preferable, the step of elution may comprise eluting the nucleic acid using a buffer with a pH between pH 5.0 to 7.0, for example between pH 5.2 to 6.8, pH 5.4 to 6.6, pH 5.6 to 6.4, pH 5.8 to 6.2.

### Nucleic Acid Amplification

In an embodiment the method further comprises a step of amplifying the nucleic acid to produce an amplified nucleic acid sample. Amplification may be performed using whole genome amplification. As used herein the term “whole genome amplification (WGA)” is term used to describe methods that amplify all DNA in a sample, often aiming to minimise amplification bias. Nucleic acid amplification methods which are suitable for use in the present method include but are not limited to techniques such as polymerase chain reaction (PCR), strand displacement amplification (SDA); rolling circle replication (RCR), nucleic acid sequence-based amplification (NASBA), ligase chain reaction (LCR), supra Q-β replicase amplification, loop-mediated isothermal amplification of DNA (LAMP), whole genome amplification including (WGA) like methods such as MDA (multiple displacement amplification) or MALBAC (multiple annealing and looping based amplification cycles), and recombinase polymerase amplification (RPA)

In the present method it is advantageous to produce long DNA fragments for subsequent sequencing and identification of organisms that are present in the sample. Further, in embodiments where identification of the microorganisms is performed at the site of sample collection, methods such as PCR may be less attractive as they require the presence of a thermocycler device, power requirements and cycling times. However, PCR provides exquisite control over the amplification conditions e.g., extension times long enough to allow long fragments to “catch up”. In these embodiments MDA or WGA may be advantageous as such methods are not necessarily a targeted PCR-like method. WGA, is designed for complex amplifications and produces much longer DNA fragments. As such in an embodiment the amplification step comprises amplifying DNA via whole genome amplification (WGA), to produce a sample of amplified DNA. In an embodiment the WGA is performed using Phi29 mediated Multiple strand Displacement Amplification.

WGA can result in the production of branched products. As such, an endonuclease is needed to ‘break’ Phi29 mediated WGA, where an unwanted product is DNA branching. DNA branching leads to physically large structures, like branches on a tree, which if directly used for sequencing on a device such as the Oxford Nanopore Technologies Minlon results in blocked sequencing pores, greatly reducing sequencing yields. To mitigate for this an endonuclease is used to break the tree like structures into shorter unbranched fragments which can be sequenced on the Minlon. In an embodiment the method comprises a step of debranching the amplified DNA, comprising contacting the amplified DNA with an endonuclease selected from SI, T7.

In an embodiment the amplification step comprises amplifying RNA via a method selected from, quantitative PCR (qPCR), real-time PCR (RT-PCR), isothermal amplification or rolling circle amplification, to produce a sample of amplified RNA.

In an embodiment the amplification step comprises a step of polyadenylating RNA. Polyadenylation of RNA ensures that all RNA can be captured by oligo d(T), ensuring specific reverse transcription of RNA, but not DNA, even with reverse transcriptases (such as Superscript IV, which is the most discriminating RT) that can transcribe both RNA and DNA.

In an embodiment the amplification step comprises converting the RNA to cDNA for further analysis. The conversion of RNA to cDNA may be performed using any suitable method known in the art, for example the extracted RNA is converted to cDNA via reverse transcription. A reverse transcriptase enzyme can be used to convert RNA to cDNA. Reverse transcriptase, also known as RNA-dependent DNA polymerase, is an enzyme used to generate complementary DNA (cDNA) from an RNA template. Specifically, the enzyme is a DNA polymerase enzyme that transcribes single-stranded RNA into DNA. This enzyme is able to synthesize a double helix DNA once the RNA has been reverse transcribed in a first step into a single-strand DNA. RNA can be reverse transcribed into cDNA using RNA-dependent DNA polymerases such as, for example, reverse transcriptases from viruses, retrotransposons, bacteria, etc. These can have RNase H activity, or reverse transcriptases can be used that are so mutated that the RNase H activity of the reverse transcriptase was restricted or is not present (e.g. MMLV-RT RNase H). Suitable reverse transcriptases include but are not limited to: AMV reverse transcriptase, MMLV reverse transcriptase, engineered MMLV reverse transcriptase. RNA-dependent DNA synthesis (reverse transcription) can also be carried by enzymes that show altered nucleic acid dependency through mutation or modified reaction conditions and thus obtain the function of the RNA-dependent DNA polymerase. In an embodiment the reverse transcriptase is Superscript IV.

In an embodiment the amplification step comprises amplifying the RNA with primers that attach a CLICK group to the amplified cDNA. The CLICK group may help to target the amplified cDNA for subsequence sequencing steps. For example, the CLICK group may interact with adaptors present on the sequencing flow cell. This targeting of the cDNA to the flow cell may achieve more efficient sequencing of the amplified cDNA.

In an embodiment the CLICK group does not comprise a copper catalysed reaction. In an embodiment the CLICK group utilises a copper-free click chemistry such as strain-promoted azide-alkyne cycloaddition (SPAAC), or inverse-electron-demand Diels-Alder (iEDDA), to attach to the adapters. In an embodiment the CLICK group is selected from a dibenzocyclooctyne group, or an azide group. In order to attach the CLICK group to the cDNA modified oligonucleotides may be used. For example 5′ DBCO modified oligos may be used for cDNA sequence library preparation methods.

### Sequencing

In an embodiment the method further comprises a step of sequencing the nucleic acid. Sequencing the nucleic acid allows the detection and identification of specific organisms, preferably pathogens within the sample. The present inventors have shown herein that using the present methods it is possible to detect low abundance organisms in the samples at a level of 1 in 200,000 or lower.

As discussed above the present methods are particularly useful for the sequencing and identification of organisms within a sample. The present methods allow long fragments (e.g., 0.5 kb to 300 kb) of nucleic acid to be successfully extracted from a sample. Sequencing of these nucleic acids provides long reads which aid in the assignment and identification of the organisms from which the nucleic acid has been obtained. These long reads provide a rich data set that allow the sequence reads to be assigned to a specific organism, the long reads can also be uniquely assigned at high confidence to a specific species or even strain. Further, wherein the sample contains a unique organism or pathogen the long reads are advantageous for genome assembly. As such the present methods are particularly useful in identifying low abundance organisms within samples.

The step of sequencing may be performed after the amplification step. However, it will be appreciated that in certain circumstances a step of amplification is not needed, and the step of sequencing may be performed after extraction of the nucleic acid.

In an embodiment the step of sequencing comprises whole genome sequencing, whole exome sequencing, targeted sequencing and/or metagenomic sequencing. In some embodiments, the sequencing includes sequencing by synthesis, sequencing by ligation, sequencing by hybridisation, sequencing by binding, and/or nanopore sequencing. In some embodiments, the sequencing by synthesis includes Illumina™ dye sequencing, single-molecule real-time (SMRT™) sequencing, or pyrosequencing. In some embodiments, the sequencing by ligation includes polony-based sequencing or SOLID™ sequencing

‘Next-Generation Sequencing’ techniques may be used to sequence the nucleic acid extracted from the sample. Particular sample preparation techniques applicable for various Next Generation sequencing approaches are known and have been extensively described, for example in manufacturer instructions for sample preparation kits available for proprietary sequencing technologies of Illumina (see, http://www.illumina.com/techniques/sequencing/ngs-library-prep.html); Pacific Biosystems (http://www.pacb.com/products-and-services/consumables/pacbio-rs-ii-consumables/sample-and-template-preparation-kits/); and Applied Biosystems (https://www.neb.com/applications/library-preparation-for-next-generation-sequencing/ion-torrent-dna-library-preparation). It will be understood by the skilled person that a variety of techniques for nucleic acid sequencing exist. These include Sanger sequencing (Sanger et al. (1977) Proceedings of the National Academy of Sciences. 74 (12) 5463-5467) and automated versions thereof, and newer technologies which are typically referred to as ‘Next Generation’ sequencing techniques (Mardis (2013) Annual Review of Analytical Chemistry. 6 287-303). Recently, nanopore sequencing, particularly the Oxford Nanopore systems (including the ‘MinION’) have seen substantial assessment and optimization for nucleic acid sequencing. The skilled person will be aware of sequencing techniques that may be used with the Oxford Nanopore MinION for example those set out in Lu et al (2016) Genomics, Proteomics & Bioinformatics. 14 (5) 265-279, which provides an overview of sequencing with the Oxford Nanopore MinION system. In an embodiment the sequencing is performed using an Oxford nanopore system such as the MiniION. It will be appreciated that portable nucleotide sequencers, such as the MinION, are particular desirable for use in the context of analysis environmental samples at the point of sample collection.

In an embodiment the input DNA amount is approximately 1 ng to 400 ng, 1 ng to 200 ng, 1 ng to 100 ng, 1 ng to 50 ng, 1 ng to 40 ng, 1 ng to 30 ng, 1 ng to 20 ng, 1 ng to 10 ng.

The methods of the present invention provides sequencing reads of >1 kb this allows unique identification of pathogens with high confidence. These long reads allow correct assignment and assembly of the sequences to allow correct identification of the organisms present in the sample. In an embodiment the sequencing reads are >0.5 kb, >0.8 kb, >1 kb, >1.2 kb, >1.4 kb, >1.6 kb, >1.8 kb, >2.0 kb, >2.2 kb, >2.4 kb, >2.6 kb, >2.8 kb, >3.0 kb, >3.2 kb, >3.4 kb, >3.6 kb, >3.8 kb, >4.0 kb. In an embodiment the sequencing reads are between 0.5 kb to 30 kb, 0.8 kb to 30 kb, 1.1 kb to 30 kb, 1.2 kb to 30 kb, 1.3 kb to 30 kb, 1.4 kb to 30 kb, 1.5 kb to 30 kb, 1.6 kb to 30 kb, 1.7 kb to 30 kb, 1.8 kb to 30 kb, 1.9 kb to 30 kb, 2.0 kb to 30 kb, 0.5 kb to 25 kb, 0.8 kb to 25 kb, 1.1 kb to 25 kb, 1.2 kb to 25 kb, 1.3 kb to 25 kb, 1.4 kb to 25 kb, 1.5 kb to 25 kb, 1.6 kb to 25 kb, 1.7 kb to 25 kb, 1.8 kb to 25 kb, 1.9 kb to 25 kb, 2.0 kb to 25 kb, 0.5 kb to 20 kb, 0.8 kb to 20 kb, 1.1 kb to 20 kb, 1.2 kb to 20 kb, 1.3 kb to 20 kb, 1.4 kb to 20 kb, 1.5 kb to 20 kb, 1.6 kb to 20 kb, 1.7 kb to 20 kb, 1.8 kb to 20 kb, 1.9 kb to 20 kb, 2.0 kb to 20 kb, 0.5 kb to 15 kb, 0.8 kb to 15 kb, 1.1 kb to 15 kb, 1.2 kb to 15 kb, 1.3 kb to 15 kb, 1.4 kb to 15 kb, 1.5 kb to 15 kb, 1.6 kb to 15 kb, 1.7 kb to 15 kb, 1.8 kb to 15 kb, 1.9 kb to 15 kb, 2.0 kb to 15 kb, 0.5 kb to 10 kb, 0.8 kb to 10 kb, 1.1 kb to 10 kb, 1.2 kb to 10 kb, 1.3 kb to 10 kb, 1.4 kb to 10 kb, 1.5 kb to 10 kb, 1.6 kb to 10 kb, 1.7 kb to 10 kb, 1.8 kb to 10 kb, 1.9 kb to 10 kb, 2.0 kb to 10 kb, 0.5 kb to 8 kb, 0.8 kb to 8 kb, 1.1 kb to 8 kb, 1.2 kb to 8 kb, 1.3 kb to 8 kb, 1.4 kb to 8 kb, 1.5 kb to 8 kb, 1.6 kb to 8 kb, 1.7 kb to 8 kb, 1.8 kb to 8 kb, 1.9 kb to 8 kb, 2.0 kb to 8 kb, 0.5 kb to 6 kb, 0.8 kb to 6 kb, 1.1 kb to 6 kb, 1.2 kb to 6 kb, 1.3 kb to 6 kb, 1.4 kb to 6 kb, 1.5 kb to 6 kb, 1.6 kb to 6 kb, 1.7 kb to 6 kb, 1.8 kb to 6 kb, 1.9 kb to 6 kb, 2.0 kb to 6 kb, 0.5 kb to 4 kb, 0.8 kb to 4 kb, 1.1 kb to 4 kb, 1.2 kb to 4 kb, 1.3 kb to 4 kb, 1.4 kb to 4 kb, 1.5 kb to 4 kb, 1.6 kb to 4 kb, 1.7 kb to 4 kb, 1.8 kb to 4 kb, 1.9 kb to 4 kb, 2.0 kb to 4 kb, 0.5 kb to 10 kb, 0.8 kb to 10 kb, 1.1 kb to 10 kb, 1.2 kb to 10 kb, 1.3 kb to 10 kb, 1.4 kb to 10 kb, 1.5 kb to 10 kb, 1.6 kb to 3 kb, 1.7 kb to 3 kb, 1.8 kb to 3 kb, 1.9 kb to 3 kb, 2.0 kb to 3 kb.

### Sample Preparation

The present methods are particularly useful in extracting nucleic acid from low abundance samples i.e., samples which contain a low amount of nucleic acid. In a preferred embodiment the sample is an environmental sample, for example an air, water, soil sample. Air samples can be particularly challenging to extract nucleic acid from and successfully sequence due to the low levels and especially low concentration of nucleic acid that may be present. In a preferred embodiment the sample is an air sample.

Where the sample to be used in the present method is an air sample, an air sampler may be used to obtain the sample. The step of air collection may be important for downstream processing, by enhancing the amount of material that is collected and subsequently extracted, this may improve profiling and sensitivity. Suitable air samplers include filter capture devices and direct to liquid devices. Examples of air sampler include the Coriolis micro, FLIR IBAC 2, InnovaPrep ACD-200 Bobcat.

Direct to liquid capture devices collect air from the environment and generate a liquid sample. The pathogens from the air are collected in the liquid sample. Suitable liquids for use in these devices include water, detergents and buffers. Suitable detergents are SDS, LiDS, Sarkosyl (N-Lauroylsarcosine), Triton X-100, Tween 20, CTAB, NONIDET P-40.

Filter capture devices pass the air sample through a filter before the collected particles are eluted to form a liquid sample. The filter may comprise charged fibres to enhance collection efficiency.

Polyvinylidene difluoride membranes are particularly preferred as they efficiently capture protein and nucleic acids.

In an embodiment the volume of air collected to produce a sample for nucleic acid extraction is between approximately 5 litres to approximately 7800 litres, 10 litres to approximately 7800 litres, 50 litres to approximately 7800 litres, 100 litres to approximately 7800 litres, 200 litres to approximately 7800 litres, 300 litres to approximately 7800 litres, 400 litres to approximately 7800 litres, 500 litres to approximately 7800 litres, 600 litres to approximately 7800 litres, 700 litres to approximately 7800 litres, 800 litres to approximately 7800 litres, 900 litres to approximately 7800 litres, 1000 litres to approximately 7800 litres, 1200 litres to approximately 7800 litres, 1400 litres to approximately 7800 litres, 1600 litres to approximately 7800 litres, 1800 litres to approximately 7800 litres, 2000 litres to approximately 7800 litres, 2200 litres to approximately 7800 litres, 2400 litres to approximately 7800 litres, 2600 litres to approximately 7800 litres, 2800 litres to approximately 7800 litres, 3000 litres to approximately 78000 litres. Preferably the volume of air collected is more than 100 litres, more than 500 litres, more than 1000 litres, more than 1500 litres, more than 2000 litres, more than 2500 litres, more than 3000 litres, more than 6000 litres, more than 9000 litres, more than 12000 litres, more than 18000 litres, more than 24000 litres, more than 27000 litres, more than 30000 litres, more than 33000 litres, more than 36000 litres, more than 39000 litres, more than 42000 litres, more than 45000 litres, more than 48000 litres, more than 51000 litres, more than 54000 litres, more than 57000 litres, more than 60000 litres, more than 63000 litres, more than 66000 litres, more than 69000 litres, more than 71000 litres, more than 74000 litres, more than 77000 litres.

In an embodiment the sample is filtered prior to performing the extraction of nucleic acid.

### Other Embodiments

An aspect of the invention relates to a method for identifying pathogens and/or allergens present in an environmental sample, the method comprising
obtaining a sample from the environment comprising one or more cells;
extracting nucleic acid from said sample by lysing the one or more cells by contacting the sample with a solution comprising a chaotropic agent; mechanically disrupting the sample comprising one or more cells; contacting the sample with magnetic particles; and contacting the sample with a non-RNA carrier molecule;
amplifying the nucleic acid; and
sequencing the nucleic acid.

An aspect of the invention relates to a method for organism profiling of an environmental sample, the method comprising
obtaining a sample from the environment comprising one or more cells;
extracting nucleic acid from said sample by lysing the one or more cells by contacting the sample with a solution comprising a chaotropic agent; mechanically disrupting the sample comprising one or more cells; contacting the sample with magnetic particles; and contacting the sample with a non-RNA carrier molecule;
amplifying the nucleic acid; and
sequencing the nucleic acid and identifying the organisms present in the environmental sample.

The methods for identifying pathogens and allergens and the methods of organism profiling in an environmental sample and microbial profiling may comprise any of the additional features which are set out herein.

In an embodiment the method of organism profiling involves profiling the whole community of the organisms present in a sample. The sample may comprise bacteria, archaea, protozoa, algae, fungi, viruses, animal, plant, and/or insect cells.

In one embodiment, the profiling method or method for identifying pathogens identifies the presence of a virus, for example but not limited to a pox virus (e.g., vaccinia virus), zika virus, smallpox virus, marburg virus, flaviviruses (e.g. Yellow Fever Virus, Dengue Virus, Tick-borne encephalitis virus, Japanese Encephalitis Virus), influenza virus (or antigens, such as F and G proteins or derivatives thereof), e.g., influenza A; or purified or recombinant proteins thereof, such as HA, NP, NA, or M proteins, or combinations thereof), parainfluenza virus (e.g., sendai virus), respiratory syncytial virus, rubeola virus, human immunodeficiency virus (or antigens, e.g., such as tat, nef, gpl20 or gpl60), human papillomavirus (or antigens, such as HPV6, 11, 16, 18), varicella-zoster virus (or antigens such as gpl, II and IE63), herpes simplex virus (e.g., herpes simplex virus I, herpes simplex virus II; or antigens, e.g., such as gD or derivatives thereof or Immediate Early protein such as ICP27 from HSV1 or HSV2), cytomegalovirus (or antigens such as gB or derivatives thereof), Epstein-Barr virus (or antigens, such as gp350 or derivatives thereof), JC virus, rhabdovirus, rotavirus, rhinovirus, adenovirus, papillomavirus, parvovirus, picomavirus, poliovirus, virus that causes mumps, virus that causes rabies, reovirus, rubella virus, togavirus, orthomyxovirus, retrovirus, hepadnavirus, hantavirus, junin virion, filovirus (e.g., ebola virus), coxsackievirus, equine encephalitis virus, Rift Valley fever virus, alphavirus (e.g., Chikungunyavirus, sindbis virus), hepatitis A virus, hepatitis B virus (or antigens thereof, for example Hepatitis B Surface antigen or a derivative thereof), hepatitis C virus, hepatitis D virus, or hepatitis E virus, coronaviruses such as SARS-COV-2, SARS-COV, or MERS-COV.

In one embodiment, the profiling method or method for identifying pathogens identifies the presence of a bacterium. Non-limiting examples of suitable bacteria which may be identified include Neisseria species, including N. gonorrhea and N. meningitidis (or antigens, such as, for example, capsular polysaccharides and conjugates thereof, transferrin-binding proteins, lactoferrin binding proteins, PiIC, adhesins); Haemophilus species, e.g., H. influenzae; S. pyogenes (or antigens, such as, for example, M proteins or fragments thereof, C5A protease, lipoteichoic acids), S. agalactiae, S. mutans; H. ducreyi; Moraxella spp, including M. catarrhalis, also known as Branhamella catarrhalis (or antigens, such as, for example, high and low molecular weight adhesins and invasins); Bordetella spp, including B. pertussis (or antigens, such as, for example, pertactin, pertussis toxin or derivatives thereof, filamenteous hemagglutinin, adenylate cyclase, fimbriae), B. parapertussis and B. bronchiseptica; Mycobacterium species, including M. tuberculosis (or antigens, such as, for example, ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (or antigens, such as, for example, colonization factors, heat-labile toxin or derivatives thereof, heat-stable toxin or derivatives thereof), enterohemorragic E. coli, enteropathogenic E. coli (or antigens, such as, for example, shiga toxin-like toxin or derivatives thereof); Vibrio spp, including V. cholera (or antigens, such as, for example, cholera toxin or derivatives thereof); Shigella spp, including S. sonnei, S. dysenteriae, S. flexnerii; Yersinia spp, including Y. enterocolitica (or antigens, such as, for example, a Yop protein), Y. pestis, Y. pseudotuberculosis; Campylobacter spp, including C. jejuni (or antigens, such as, for example, toxins, adhesins and invasins) and C. coli; Salmonella spp, including S. typhi, S. paratyphi, S. choleraesuis, S. enteritidis, S. typhimurium, and S. dysenteriae; Listeria species, including L. monocytogenes; Helicobacter spp, including H. pylori (for example urease, catalase, vacuolating toxin); Pseudomonas spp, including P. aeruginosa; Staphylococcus species, including S. aureus, S. epidermidis; Proteus species, e.g., P. mirabilis; Enterococcus species, including E. faecalis, E. faecium; Clostridium species, including C. tetani (or antigens, such as, for example, tetanus toxin and derivative thereof), C. botulinum (or antigens, such as, for example, botulinum toxin and derivative thereof), C. difficile (or antigens, such as, for example, Clostridium toxins A or B and derivatives thereof), and C. perfringens; Bacillus species, including B. anthracis (or antigens, such as, for example, anthrax toxin and derivatives thereof), B. cereus, B. circulans and B. megaterium; Corynebacterium species, including C. diphtheriae (or antigens, such as, for example, diphtheria toxin and derivatives thereof); Borrelia species, including B. burgdorferi (for example OspA, OspC, DbpA, DbpB), B. garinii (or antigens, such as, for example, OspA, OspC, DbpA, DbpB), B. afzelii (for example OspA, OspC, DbpA, DbpB), B. andersonii (or antigens, such as, for example, OspA, OspC, DbpA, DbpB), B. hermsii; Ehrlichia species, including E. equi and the agent of the Human Granulocytic Ehrlichiosis; Rickettsia spp, including R. rickettsii; Chlamydia species, including C. trachomatis (or antigens, such as, for example, MOMP, heparin-binding proteins), C. pneumoniae (for example MOMP, heparin-binding proteins), C. psittaci; Leptospira species, including L. interrogans; Streptococcus species, such as S. pyogenes, S. agalactiae, S. pneumonia; Treponema species, including T. pallidum (or antigens, such as, for example, the rare outer membrane proteins), T. denticola, and T. hyodysenteriae.

In one embodiment, the profiling method or method for identifying pathogens identifies the presence of a parasite. Non-limiting examples of suitable parasite which may be identifies include Plasmodium species, including P. falciparum; Toxoplasma species, including T. gondii (or antigens, such as, for example SAG2, SAG3, Tg34); Entamoeba species, including E. histolytica; Babesia species, including B. microti; Trypanosoma species, including T cruzi; Giardia species, including G. lamblia; Leshmania species, including L. major; Pneumocystis species, including P. carinii; Trichomonas species, including T. vaginalis; and Schisostoma species, including S. mansoni.

In another embodiment, the profiling method or method for identifying pathogens identifies the presence of a fungus (for example Cryptococcus neoformans or Aspergillus). In another embodiment, the profiling method or method for identifying pathogens identifies the presence of a protozoan. Suitable protozoans which may be detected include, without limitation, protests (unicellular or multicellular), e.g., Plasmodium falciparum, and helminths, e.g., cestodes, nematodes, and trematodes.

The organism profiling method may comprise profiling pathogens and allergens. Allergens may include airborne allergens such as pollen, animal dander, dust mites and/or molds.

In a preferred embodiment the environmental sample is an air sample. The pathogens that may be identified include microorganisms such as bacteria, archaea, protozoa, algae, fungi, viruses.

The methods described herein may be combined with bioinformatics or computer implemented approaches for the identification of pathogens or microorganisms. In particular identification of the organisms present in the sample may comprise bioinformatics methods to identify the species of the organisms present. Bioinformatics approaches and databases are known in the art which can be used in the identification of organisms from sequencing data, such as the Basic Local Alignment Tool (BLAST). BLAST is described for example in “Basic Local Alignment Search Tool” by Altschul et al published in Journal of Molecular Biology 1990 Oct. 5; 215 (3): 403-10. BLAST implements a seed and extend algorithm to find matches between the query sequence and target sequences in the database.

An aspect of the present invention relates to a kit for the extraction of nucleic acid, comprising a solution comprising a chaotropic agent, magnetic particles, a non-RNA carrier and optionally instructions for use. The instructions for use may set out how to perform one or more of the methods described herein.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The term “comprising” or “comprises” where used herein means including the component(s) specified but not to the exclusion of the presence of other components. The term “consisting essentially of” or “consists essentially of” means including the components specified but excluding other components except for materials present as impurities, unavoidable materials present as a result of processes used to provide the components and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

The invention is further illustrated in the following non-limiting examples.

### EXAMPLES

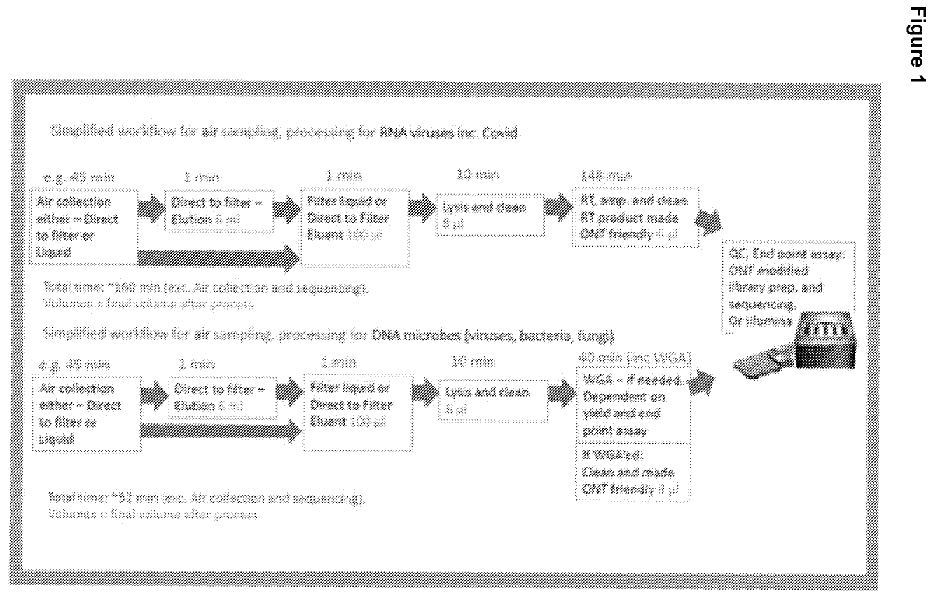

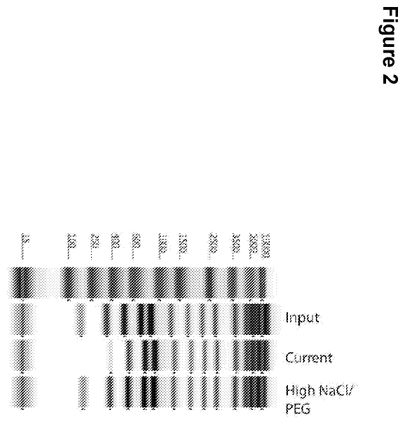

The inventors have developed a method for extracting a nucleic acid from a sample comprising one or more cells comprising nucleic acid. A flowchart showing the steps of extracting and sequencing nucleic acid from samples comprising RNA viruses and DNA microbes (viruses, bacteria and fungi) is shown in FIG. 1.

For both RNA and DNA viruses, the initial air collection, elution, lysis and clean-up steps are as follows:

### Prepare:

Swinney filter holder (13 mm diameter) loaded with 0.1 μm (or other pore size) PVDF filter membrane
Paramagnetic bead nucleic acid binding buffer (2 ml buffer: 20 μl 1M Tris-HCl PH 7.5, 4 μl 0.5M EDTA, 640 μl 5M NaCl, 440 μl 50% w/v PEG-8000, 896 μl DNase/RNase free water)
Lysis tube and beads e.g. PowerBead Pro Tube (DNeasy PowerSoil Pro Kit) tube with a reduced amount of 0.25 g PowerBead particles
   Extraction Protocol (Assuming Use of Bobcat and Elution with Bobcat Foaming Buffer):
1) Sample air, for example 45 minutes using Innovaprep Bobcat continuous mode.
2) Elute in Bobcat collection tube with Bobcat foaming buffer.
3) Take up Bobcat eluate using a 10 ml syringe and filter the eluate through the PVDF membrane.
4) Transfer the PVDF filter to the Lysis tube and beads e.g. PowerBead Pro Tube and add 100 μl CD1 buffer (DNeasy PowerSoil Pro Kit; contains guanidinium).
5) Grind for 5 minutes at 1,500 rpm (=25/s).
6) Centrifuge the sample on max speed (>14 k RPM) for 1 minute.
7) 1 μl of nuclease free GenElute LPA (25 mg/ml solution in water) was added. Transfer the entire supernatant to a 1.5 ml tube and perform a 1× paramagnetic (e.g. SPRI) bead cleanup (100 μl paramagnetic beads, 2× wash in 200 μl 70% v/v EtOH) and elute in 8 μl DNase/RNase free water. These steps recover both DNA and RNA. Then proceed with DNA and RNA library preparation protocols. The RNA and DNA may be separately tagged (tracked) from the shared extract to the sequencer.

Retain beads by adding 20 μl paramagnetic binding buffer for later recycling.

For RNA viruses, air collection, elution, lysis and clean-up are followed by reverse transcription, amplification and sequencing. The following pipeline protocol has been developed by the inventors for RNA viruses:

RNA Amplification and cDNA Sequencing Library Protocol
1) Polyadenylation of RNA: Mix 8 μl of sample with 2 μl 10× Poly(A) polymerase buffer, 2 μl ATP, 7 μl nuclease-free water, and 1 μl Poly(A) polymerase, and incubate at 37° C. for 5 min. [polyadenylation ensures all RNAs are captured, even those without A tails. The addition of polyadenylation also adds enzyme selection for RNA]
2) Clean up using the standard 1×SPRI bead protocol and elute in 4 μl water.
3) Reverse transcription: Mix 4 μl of the sample with 1 μl ISPCR-OdTVN primer (2 μM), incubate at 70° C. for 5 min, and snap-cool on ice. Add 0.8 μl water, 1.5 μl PEG 8000 (50%), 2 μl 5× Superscript IV buffer, 0.4 μl dNTPs (100 mM), 0.2 μl ISPCR-TSO primer (100 μM), and 0.1 μl Superscript IV, mix, and incubate at 50° C. for 30 min, followed by 5 min. at 85° C. [the inventors have found that the use of an oligo dT primer with Superscript IV results in 10× increased specificity for RNA compared to DNA]
4) Set up 5 PCR reactions by mixing 2 μl of the RT reaction with 14.74 μl nuclease-free water, 4.4 μl 10× Q5 buffer, 0.44 μl dNTPs (10 mM), 0.2 μl ISPCR primer (10 UM), and 0.22 μl Q5 High Fidelity DNA polymerase. Cycle the reactions using the program 98° C. 3 min, 25× [98° C. 20 sec, 67° C. 15 sec, 72° C. 2 min], 72° C. 5 min, 4° C. forever on a ProFlex thermal cycler (Thermo Fisher).
5) Pool the 5 PCR reactions, clean up using the standard 1×SPRI bead protocol, and elute in 20 μl water.
6) Quantify 2 μl with Qubit dsDNA High Sensitivity and proceed with library preparation using 200 fmol DNA and the ONT Ligation Sequencing (SQK-LSK009) kit.

Notably, by using two enzymatic steps (which are RNA specific) results in little DNA bleed through into RNA libraries such that DNA and RNA pipelines can be run in parallel.

The total time required for steps 1-5 is 142 min. However, the inventors have noted that it is possible to reduce the total time required for steps 1-5 down to 60 min by reducing the reverse transcription incubation time, and using faster DNA polymerases with reduced extension time and/or lower number of PCR cycles. The inventors also note that it is possible to reduce the library preparation time from 30 min using the ONT Ligation Sequencing kit to just 1 min using CLICK chemistry, e.g. using DBCO-modified primers in the PCR amplification step, and the RAP (rapid adapter mix) from the ONT Rapid Sequencing kit to prepare the sequencing library.

For DNA viruses, air collection, elution, lysis and clean-up are followed by optional WGA and sequencing. The following pipeline protocol has been developed by the inventors for DNA viruses:

### DNA Whole Genome Amplification (WGA) and Endonuclease Treatment Protocol:

WGA—for example Thermo EquiPhi or Qiagen Repli-G (multiple stranded displacement amplification) modified protocol:
1) Combine 3.75 μl DNA with 0.25 μl WGA Repli-G Ultrafast neat DLB buffer (alkali treatment), mix well and incubate the reaction for 3 minutes to denature the extracted gDNA making it a good template when neutralised with Repli-G Ultrafast neat Stop solution. [N. B. The alkali treatment would hydrolyse RNA]
2) After 3 minutes add 0.40 μl Repli-G Ultrafast neat Stop solution and mix well.
3) Add 16 μl Repli-G Ultrafast reaction buffer and 1 μl Repli-G Ultrafast polymerase to the reaction, mix well and incubate for 90 minutes at 30° C.
4) Clean the reaction with a 1× paramagnetic bead cleanup by re-using the retained beads from the extraction step (20 μl beads, 2× wash in 200 μl 70% v/v EtOH), elute in 17 μl DNase/RNase free water. Retain paramagnetic beads by adding 20 μl binding buffer for later recycling.
5) Add 4 μl 5× Thermo Scientific S1 nuclease buffer and 1 μl (100 U/μl) Thermo Scientific S1 nuclease to the clean WGA reaction and incubate for 15 minutes at 37° C.
6) Perform a final 1× cleanup by re-using the above prepared paramagnetic beads e.g. SPRI (20 μl SPRI beads, 2× wash in 200 μl 70% v/v EtOH), elute in 9 μl 1× TE buffer, quantify 1 μl using Qubit 2.0 Broad Range reagents and dilute to 400 ng DNA to 7.5 μl in 1× TE buffer. Proceed with the 7.5 μl to the ONT Rapid Barcoding Sequencing Kit (SQK-RBK004).

### Example 1—Nucleic Acid Extraction Methods

The present inventors have developed an optimised method to efficiently capture RNA and DNA, and which can be used for simultaneous extraction of DNA and RNA whilst avoiding the use of carrier RNA. This resulted in improvements in DNA extraction allowing measurable amounts of material obtained (from undetectable levels to 3-15 ng).
Optimised lysis volume and bead weight to improve molecular weight (i.e. length of nucleic acid molecules extracted) of DNA extracted and yield.
Showed robustness of methodology through consistent Femto Pulse electropherograms.
Replaced commercial beads with homebrew recipe, reducing cost and allowing same beads to be reused throughout the pipeline.
Combined lysis and concentration steps by precipitating nucleic acids onto beads to enable enzymatic reactions to proceed on the beads without a prior elution step.
Reworked DNA precipitation and elution times.
Cut bead beating times by 90%.

These optimisations are detailed further below.

### Preparation of Home-Brew Magnetic Beads

Beads are Sera-Mag Carboxylate-Modified Magnetic Particles (Hydrophylic) from GE lifesciences and the inventors use 100 μl in 2 mls of PEG solution (see below).

2 ml PEG Solution contains

| | | |
|---|---|---|
| 20 | μl | 1M TRIS-HCl |
| 4 | μl | 0.5M EDTA |
| 640 | μl | 5M NaCL |
| 440 | μl | 50% w/v PEG 8000 |
| 896 | μl | DNase free H₂O |

### Protocol

Take 100 μl of beads and add to a 2 ml Eppendorf tube.
Centrifuge then place on a Magnetic Particle Concentrator (MPC) to pellet the beads.
Discard the supernatant.
Remove tubes with beads from the MPC
Add 1 ml 10 mM Tris and vortex to resuspend the beads
Centrifuge then place on a Magnetic Particle Concentrator (MPC) to pellet the beads.
Discard the supernatant.
Remove tubes with beads from the MPC
Add 1 ml 10 mM Tris and vortex to resuspend the beads
Centrifuge then place on a Magnetic Particle Concentrator (MPC) to pellet the beads.
Discard the supernatant
Remove tubes with beads from the MPC
Add 2 mls of the PEG solution and vortex.
Store beads in Fridge for up to 4 weeks.

### Testing the Effect of Beat Beating Times on DNA Yield

1 μl of Zymo Mock cells were added to 100 μl of CD1 lysis buffer and 250 mgs beads (taken from Qiagen DNeasy PowerSoil Pro Kit) and beaten for 0, 1, 2, 3, 4, and 5 minutes. The results (Table 1) showed that 5 minutes beat beating is important. There will be some lysis without the need for bead beating as gram negative cells should be easily burst in the presence of a lysis solution. However, gram positive bacteria and spores will need more aggressive bead beating.

TABLE 1
Effect of beat beating times on DNA yield

| Beat beating time | DNA concentration |
|---|---|
| (minutes) | (ng/μl) |
| 0 | 6.66 |
| 1 | 6.25 |
| 2 | 6.30 |
| 3 | 6.59 |
| 4 | 6.80 |
| 5 | 8.30 |

Testing the Effect of Temperature on DNA Precipitation onto Magnetic Beads

A cool block was cooled to 10° C. then parallel precipitations performed onto beads for 5 minutes either at 10 or 22° C. using 1 μl of Zymo Mock cells added to 100 μl of CD1 lysis buffer and 250 mgs beads (taken from Qiagen DNeasy PowerSoil Pro Kit) and beaten for 5 minutes using a Genogrinder (from Spex Sampleprep) or Qiagen TissueLyser. The precipitated DNA was washed twice with 70% ethanol and then eluted for 5 m at room temperature. DNA concentration was measured using the Qubit High Sense assay. The results (Table 2) suggest that there is no appreciable difference between precipitating beads at 22 and 10° C.

TABLE 2
Effect of temperature on DNA precipitation onto magnetic beads

| Precipitation temperature (° C.) | DNA concentration (ng/μl) |
|---|---|
| 10 | 5.1 |
| 22 | 4.8 |

Testing the Effect of Temperature of DNA Elution from Magnetic Beads

A peltier heat/cooling block was preheated to 37° C. then parallel precipitations performed onto beads for 5 minutes using 1 μl of Zymo Mock cells added to 100 μl of CD1 lysis buffer and 250 mgs beads (taken from Qiagen DNeasy PowerSoil Pro Kit) and beaten for 5 minutes using a Genogrinder (from Spex Sampleprep) or Qiagen TissueLyser. The precipitated DNA was washed twice with 70% ethanol and then eluted for 5 m at either 22 or 37° C. DNA concentration was measured using the Qubit High Sense assay. The results (Table 3) show no appreciable difference between eluting DNA from beads at 22 and 37° C.

TABLE 3
Effect of temperature of DNA elution from magnetic beads

| Elution | DNA |
|---|---|
| temperature | concentration |
| (° C.) | (ng/μl) |
| 22 | 7.44 |
| 37 | 7.58 |

Testing the Effect of Reducing the Time of DNA Precipitation onto Magnetic Beads

Parallel precipitations performed onto beads for 1, 3 or 5 minutes using 1 μl of Zymo Mock cells added to 100 μl of CD1 lysis buffer and 250 mgs beads (taken from Qiagen DNeasy PowerSoil Pro Kit) and beaten for 5 minutes using a Genogrinder (from Spex Sampleprep) or Qiagen TissueLyser. The precipitated DNA was washed twice with 70% ethanol and then eluted for 5 minutes at room temperature. DNA concentration was measured using the Qubit High Sense assay. Results (Table 4) surprisingly show that we can reduce the precipitation time to 3 mins which could save up to 9 minutes in the pipeline.

TABLE 4
Effect of time of DNA precipitation

| Precipitation | DNA |
|---|---|
| time | concentration |
| (minutes) | (ng/μl) |
| 1 | 0.81 |
| 3 | 6.31 |
| 5 | 6.1 |

Testing the Effect of Reducing DNA Elution Time from Magnetic Beads

Parallel precipitations performed onto beads for 3 minutes using 1 μl of Zymo Mock cells added to 100 μl of lysis buffer and 250 mgs beads and beaten for 5 minutes. The precipitated DNA was washed twice with 70% ethanol and then eluted for 2, 3.5 or 5 minutes at room temperature. DNA concentration was measured using the Qubit High Sense assay. The results (Table 5) show that the elution time can unexpectedly be reduced to 2 mins which could save up to 9 minutes in the pipeline.

TABLE 5
Effect of reduction of DNA elution time from beads

| Elution | DNA |
|---|---|
| time | concentration |
| (minutes) | (ng/μl) |
| 2 | 6.51 |
| 3.5 | 6.08 |
| 5 | 6.35 |

### Testing the Effect of Reduced Precipitation and Elution Time on Sequencing

The above results suggest that up to 5 mins per bead based clean up could be saved. To check that the clean-up is unaffected and make sure that there are no impurities that affect sequencing the inventors needed to compare sequencing a standard clean up sample versus the reduced time clean up.

Parallel precipitations performed onto beads for 3 or 5 minutes using 1 μl of Zymo Mock cells added to 100 μl of lysis buffer and 250 mgs beads and beaten for 5 minutes. The precipitated DNA was washed twice with 70% ethanol and then eluted for 2 or 5 minutes respectively at room temperature. DNA concentration was measured using the Qubit High Sense assay. For sequencing 20 ng of DNA was used in a half reaction RAD04 rapid library.

DNA yield was around 12% lower for the reduced time clean up but interestingly, on sequencing, read N50 was about 20% higher for the first hour which may help with classification (Tables 6 and 7). This suggests that extra couple of minutes precipitation onto beads is allowing smaller molecules to be captured onto the beads. With read N50 being slightly longer the time to 52 k reads is slightly longer but this is to be expected.

TABLE 6
Effect of reduced precipitation and elution on DNA extraction

| | DNA |
|---|---|
| Clean up | concentration |
| method | (ng/μl) |
| 5 min ppt, 5 min elute | 11.2 |
| 3 min ppt, 2 min elute | 9.86 |

TABLE 7
Effect of reduced precipitation and elution on sequencing

| Clean up | Time to 52k passed |
|---|---|
| method | filter reads (m) |
| 5 min ppt, 5 min elute | 55 |
| 3 min ppt, 2 min elute | 58 |

### Comparison of Bead Recovery Using Different Magnets, Different Paramagnetic Beads and Different Solutions

The inventors tested two magnet racks: Dynal magnet and DynaMag2 with the following magnetic beads, silica coated beads are marked with the asterisk: (1) Appleton Woods AppMag PCR Clean Up beads, (2) Axygen AxyPrep Mag PCR Clean up beads, (3) HomeBrew beads, (4) ABI ThermoNucleic acid binding beads*, (5) MagBio HighPrep PCR clean up beads, (6) Nimagen AmpliCleanMagnetic beads, (7) ZymoBiomics MagBinding beads*, (8) Beckman Coulter Ampure XP beads.

The inventors performed the mock clean ups using 10 μl of beads treated initially with 20 μl of water. Then 20 μl of a suitable buffer (either SPRI or PB) was added to the washed beads. Then each reaction was washed twice with 200 μl of 70% ethanol and eluted in 10 μl water. The time of bead recovery of each step for each type of the beads on both magnets was measured and the solutions were kept for further for spectrophotometric measurements at 260 nm (whilst we added no DNA, released/lost bead particles will absorb or scatter the incident light, thus this measures bead loss). Only the original bead solution was discarded, however the recovery time was still measured. All reactions were quantified using Nanodrop for DNA wavelength measurement option with the device calibrated with a suitable solution used (water, SPRI, PB buffer or ethanol). The results are presented in Table 8 below.

TABLE 8
Comparison of bead recovery using different magnets, beads and solutions

| | | | | Nanodrop | | |
|---|---|---|---|---|---|---|
| | | Bead recovery time | | Measurement (ng/μl) | | |
| | Step of bead | Dynal | | Dynal | | Nanodrop Measure |
| Beads used | treatment | magnet | DynaMag2 | magnet | DynaMag2 | of pure solutions |
| Appleton Woods | Own solution | 2 m 06 s | 1 m 54 s | | | |
| | Water wash | 20 s | 18 s | 0.6282 | −0.2117 | −0.09035 to −0.4118 |
| | SPRI buffer | 1 m 11 s | 1 m 12 s | −2.343 | −1.485 | −1.4840 to 0.3010 |
| | Ethanol 1 | 8 s | 5 s | −0.6737 | −1.147 | −0.3083 to −0.7045 |
| | Ethanol 2 | 8 s | 10 s | −2.088 | −2.759 | |
| | Elution | 14 s | 15 s | −0.1315 | 0.3345 | −0.09035 to −0.4118 |
| | Total time | 4 m 07 s | 3 m 54 | | | |
| Axygen | Own solution | 2 m 50 s | 2 m 18 s | | | |
| | Water wash | 20 s | 16 s | 0.5719 | −0.3925 | −0.09035 to −0.4118 |
| | SPRI buffer | 1 m 03 s | 1 m 18 s | −3.2020 | −1.344 | −1.4840 to 0.3010 |
| | Ethanol 1 | 1 s | 1 s | −0.2634 | −0.2851 | −0.3083 to −0.7045 |
| | Ethanol 2 | 15 s | 5 s | −1.6900 | −0.6897 | |
| | Elution | 17 s | 14 s | 0.1189 | −0.6315 | −0.09035 to −0.4118 |
| | Total time | 4 m 46 s | 4 m 12 s | | | |
| HomeBrew | Own solution | 11 s | 6 s | | | |
| | Water wash | 17 s | 12 s | 7.628 | 5.648 | −0.09035 to −0.4118 |
| | SPRI buffer | 45 s | 32 s | 1.330 | −0.9203 | −1.4840 to 0.3010 |
| | Ethanol 1 | 26 s | 27 s | 0.6534 | −0.7842 | −0.3083 to −0.7045 |
| | Ethanol 2 | 26 s | 28 s | 0.5333 | −1.265 | |
| | Elution | 15 s | 10 s | 3.663 | 1.678 | −0.09035 to −0.4118 |
| | Total time | 2 m 20 s | 1 m 55 s | | | |
| ABI Thermo | Own solution | 26 s | 17 s | | | |
| Scientific* | Water wash | 25 s | 26 s | 7.4310 | 8.488 | −0.09035 to −0.4118 |
| | PB buffer | 1 m 23 s | 1 m 22 s | 4.0610 | 1.217 | 0.0669 to −0.6998 |
| | Ethanol 1 | 7 s | 4 s | −0.9792 | −0.6491 | −0.3083 to −0.7045 |
| | Ethanol 2 | 3 s | 7 s | −0.6908 | −1.4880 | |
| | Elution | 21 s | 15 s | 0.5888 | 1.504 | −0.09035 to −0.4118 |
| | Total time | 2 m 45 s | 2 m 31 s | | | |
| MagBio | Own solution | 1 m 36 s | 1 m 45 s | | | |
| | Water wash | 17 s | 15 s | 0.4512 | −0.4286 | −0.09035 to −0.4118 |
| | SPRI buffer | 1 m 07 s | 53 s | −8.875 | −1.394 | −1.4840 to 0.3010 |
| | Ethanol 1 | 3 s | 4 s | 23.19 | −1.236 | −0.3083 to −0.7045 |
| | Ethanol 2 | 3 s | 10 s | −0.9411 | −0.7001 | |
| | Elution | 15 s | 12 s | 0.1116 | 0.0534 | −0.09035 to −0.4118 |
| | Total time | 3 m 21 s | 3 m 19 s | | | |
| AmpliClean | Own solution | 2 m 12 s | 2 m 23 s | | | |
| | Water wash | 21 s | 22 s | 0.2299 | −0.1402 | −0.09035 to −0.4118 |
| | SPRI buffer | 1 m 16 s | 1 m 12 s | −2.980 | −2.511 | −1.4840 to 0.3010 |
| | Ethanol 1 | 5 s | 5 s | −1.263 | −0.4504 | −0.3083 to −0.7045 |
| | Ethanol 2 | 27 s | 28 s | −1.031 | −1.553 | |
| | Elution | 15 s | 14 s | −0.1159 | −0.1278 | −0.09035 to −0.4118 |
| | Total time | 4 m 36 s | 4 m 44 s | | | |
| ZymoBiomics* | Own solution | 1 s | 1 s | | | |
| | Water wash | 6 s | 7 s | 72.23 | 32.50 | −0.09035 to −0.4118 |
| | PB buffer | 10 s | 12 s | 2.747 | 0.1987 | 0.0669 to −0.6998 |
| | Ethanol 1 | 11 s | 7 s | −0.1458 | −0.4335 | −0.3083 to −0.7045 |
| | Ethanol 2 | 11 s | 9 s | −0.3100 | −0.6860 | |
| | Elution | 9 s | 1 s | 0.1549 | −0.4890 | −0.09035 to −0.4118 |
| | Total time | 48 s | 37 s | | | |
| AmpureXP | Own solution | 2 m 06 s | 2 m 04 s | | | |
| | Water wash | 20 s | 17 s | 0.3260 | −0.7823 | −0.09035 to −0.4118 |
| | SPRI buffer | 1 m 21 s | 1 m 08 s | −2.943 | −2.633 | −1.4840 to 0.3010 |
| | Ethanol 1 | 11 s | 4 s | −1.160 | −0.4877 | −0.3083 to −0.7045 |
| | Ethanol 2 | 13 s | 25 s | −1.612 | −4.395 | |
| | Elution | 14 s | 15 s | −0.0439 | 0.6980 | −0.09035 to −0.4118 |
| | Total time | 4 m 25 s | 4 m 13 s | | | |

The inventors found that there were differences in beads recovery times and sample purity between the two tested magnets with DynaMag2 presenting quicker recovery (clumping) times (from a few seconds to half a minute) and higher DNA yield. Only in the case of Nimagen AmpliClean Magnetic beads the Dynal magnet performed slightly better by a few seconds and lower DNA (bead particle) readings. There were bigger differences between the different types of beads with ZymoBiomics and HomeBrew being the fastest (less than 1 minute and just above 1 minute recovery time, respectively) and Axygen and AmpliClean being the slowest (both over 4 minutes). Interestingly the slowest beads presented the highest DNA yield suggesting the bead loss was the smallest, and the fastest beads gave lower DNA yield levels.

Tube Based Comparison of the Efficiency of 2 Magnets in Magnetic Beads Recovery with the Use of DNA

The inventors tested two magnet racks: Dynal magnet and DynaMag2 with the following magnetic beads, silica coated beads were marked with an asterisk: (1) Axygen AxyPrep Mag PCR Clean up beads, (2) HomeBrew beads, (3) Nimagen AmpliClean Magnetic beads, and (4) ZymoBiomics MagBinding beads*. The inventors performed the clean ups using 13.2 ng Bioline 1 kb Hyperladder diluted in 10 μl water (original ladder diluted 1/100 and quantified using Qubit 2.0 HighSensitivity reagents). To each ladder dilution the inventors added 10 μl beads, to the silica coated particles we further added 20 μl Buffer PB as binding buffer. The inventors incubated the reactions for 5 minutes on room temperature, washed each reaction twice with 200 μl 70% ethanol and eluted for 5 minutes in 10 μl water. All reactions were quantified using Qubit 2.0 HighSensitivity reagents. The results are presented in Table 9 below.

TABLE 9
Sample purification time and sample purity measured for each
step, each type of beads and each type of magnet racks used

| | Step of bead | Bead recovery time | | Qubit Measurement (ng/μl) | |
|---|---|---|---|---|---|
| Beads used | treatment | Dynal magnet | DynaMag2 | Dynal magnet | DynaMag2 |
| Axygen | Own solution | 2 m 30 s | 2 m 30 s | | |
| | Water wash | 24 s | 22 s | | |
| | SPRI buffer | 55 s | 52 s | 1.48 | 1.51 |
| | Ethanol 1 | 5 s | 6 s | <50 | <50 |
| | Ethanol 2 | 3 s | 3 s | <50 | <50 |
| | Elution | 23 s | 23 s | 8.76 | 9.20 |
| | Total time | 4 m 20 s | 4 m 16 s | | |
| HomeBrew | Own solution | 10 s | 10 s | | |
| | Water wash | 18 s | 20 s | | |
| | SPRI buffer | 48 s | 40 s | 0.904 | 0.830 |
| | Ethanol 1 | 12 s | 5 s | <50 | <50 |
| | Ethanol 2 | 15 s | 6 s | <50 | <50 |
| | Elution | 22 s | 22 s | 11.2 | 12.2 |
| | Total time | 2 m 05 s | 1 m 43 s | | |
| AmpliClean | Own solution | 2 m 10 s | 2 m 12 s | | |
| | Water wash | 24 s | 22 s | | |
| | SPRI buffer | 58 s | 1 m | 1.35 | 1.49 |
| | Ethanol 1 | 5 s | 4 s | <50 | <50 |
| | Ethanol 2 | 3 s | 2 s | <50 | <50 |
| | Elution | 22 s | 22 s | 7.18 | 9.30 |
| | Total time | 4 m 05 s | 4 m 02 s | | |
| ZymoBiomics* | Own solution | 1 s | 1 s | | |
| | Water wash | 5 s | 5 s | | |
| | PB buffer | 8 s | 10 s | 0.674 | 0.382 |
| | Ethanol 1 | 12 s | 12 s | <50 | <50 |
| | Ethanol 2 | 10 s | 8 s | <50 | <50 |
| | Elution | 5 s | 4 s | 9.78 | 10.6 |
| | Total time | 41 s | 40 s | | |

The inventors found the DynaMag2 rack more effective than Dynal with quicker bead recovery time and higher total DNA yield in the final elution than in the solutions from preceding steps, suggesting smaller bead loss on the way. Once again HomeBrew beads proved to be the most efficient both regarding DNA yield and time efficiency, despite the type of magnet used. DNA presence does not seem to affect the behaviour of beads and thus their losses.

Testing the Effect of Increased NaCl and PEG8000 Concentrations in SPRI Bead DNA Binding buffer on DNA and RNA clean-up

The inventors sought to investigate whether increasing the concentration of NaCl and PEG 8000 in the DNA binding buffer could recover lower molecular weight RNA and DNA fragments. DNA binding buffers with different concentrations of NaCl and PEG 8000 were prepared as detailed in Table 10. 5 μl SPRI beads were washed 3× in nuclease-free water and suspended in 100 μl DNA binding buffer. To assess the effect on recovery of low molecular weight DNA and RNA, 5 μl Bioline 1 kb DNA Hyperladder (590 ng) and 5 μl of the Qubit RNA BR Standard #2 (500 ng) were diluted with nuclease-free water to a final volume of 20 μl and used in separate tubes for each DNA binding buffer.

TABLE 10
Composition of DNA binding buffers tested

| | | |
|---|---|---|
| Buffer | Current | High NaCl/PEG |

| | | | | |
|---|---|---|---|---|
| Tris-HCl pH 8.0 | 10 | mM | 10 | mM |
| EDTA | 1 | mM | 1 | mM |
| NaCl | 1.6 | M | 2.5 | M |

| | | |
|---|---|---|
| PEG 8000 | 11% | 20% |

20 μl SPRI bead solution was added to each tube, incubated for 5 min at room temperature, and pelleted on a magnetic rack. Bead pellets were washed twice in freshly made 70% ethanol and eluted in 10 μl nuclease-free water. The eluate was analysed using Qubit dsDNA and RNA, and D5000 and RNA screen tapes. The current DNA binding buffer is efficient in selecting fragments longer than 500 bp, which results in a lower total yield both for DNA and RNA. Increasing the concentration of NaCl and PEG 8000 increases recovery of lower molecular weight DNA and RNA fragments (and total yields). Thus, using a DNA binding buffer with higher NaCl and PEG 8000 concentrations may be useful for extractions and clean-up where shorter (RNA) fragments should be retained.

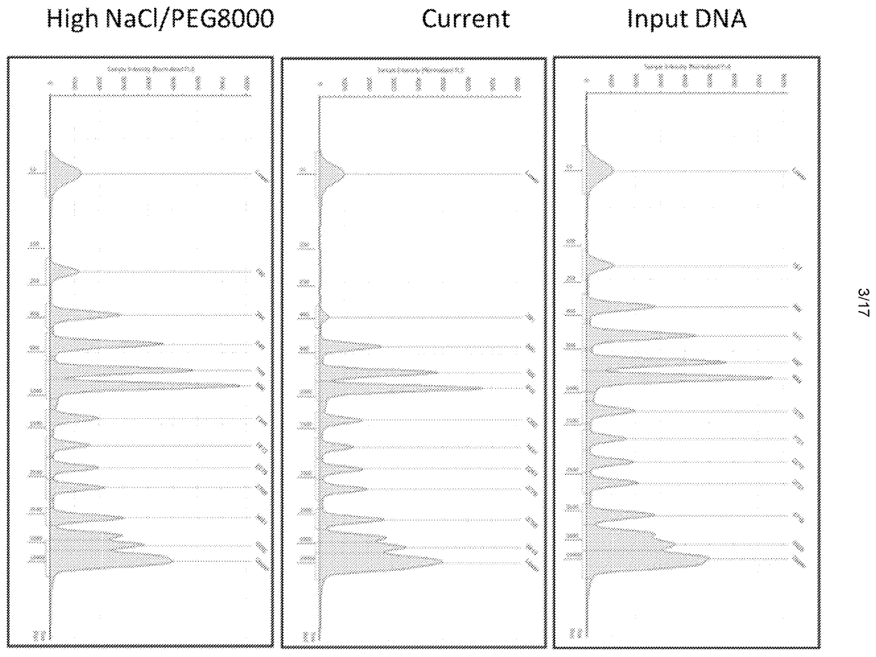

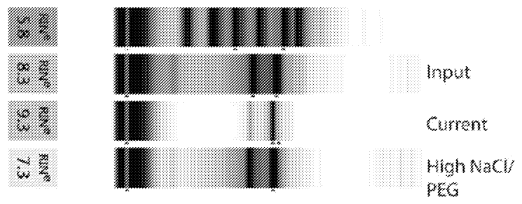

As shown in Table 11 and FIG. 2, increased concentrations of NaCl and PEG 8000 results in increased recovery of lower molecular weight DNA and RNA molecules.

TABLE 11
Quantification of recovered DNA and RNA

| Buffer | DNA | % recov. | RNA | % recov. |
|---|---|---|---|---|
| Current | 427 ng | 72% | 126 ng | 25% |
| High NaCl/PEG 8000 | 580 ng | 98% | 368 ng | 74 |

### Consistency in Yield Across Multiple Repetitions of Magnetic Bead DNA Clean-Up

The inventors next tested the variability in DNA yield in technical replicates and independent experiments of magnetic bead-based DNA clean-up a key component of the automated molecular biology pipeline.

In this experiment, Bioline 1 kb DNA Hyperladder was cleaned up using the following magnetic bead clean-up protocols:
1) SeraMag SPRI beads with low NaCl/PEG8000 DNA binding buffer (standard-ST);
2) SeraMag SPRI beads with high NaCl/PEG 8000 DNA binding buffer (high Na/PEG);
3) AmpliClean beads;
4) MagBio beads.

Three technical replicates were performed for each protocol, and the whole experiment repeated on 3 separate days, resulting in 9 data points for each bead protocol.

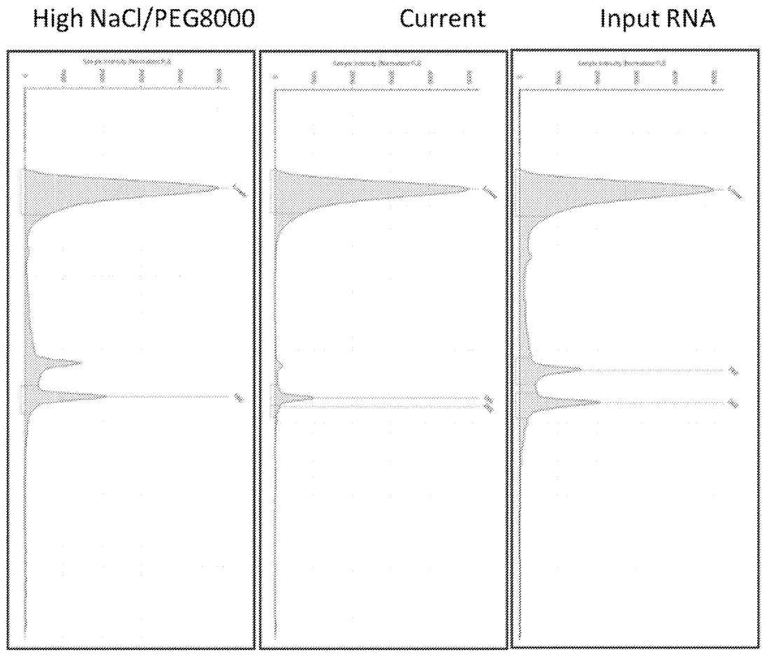

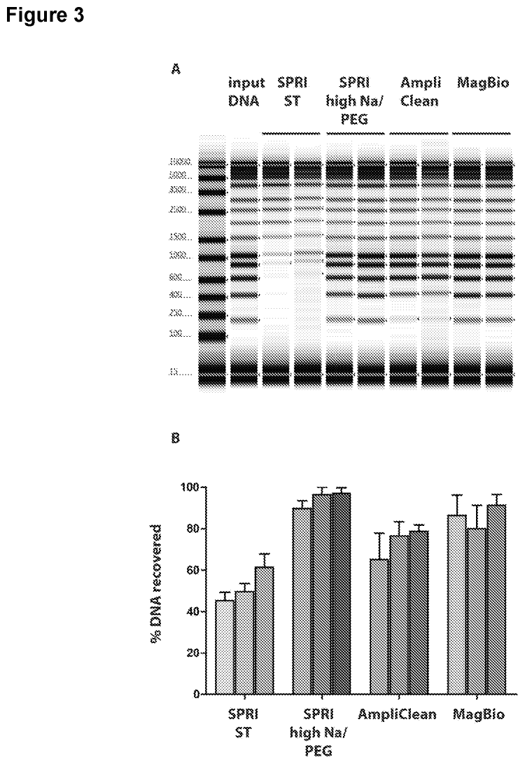

Briefly, 2 μl Bioline 1 kb DNA Hyperladder was diluted 1:5, and 10 μl of diluted ladder was used in 1× bead clean-up experiments. The DNA-bead mix was left to incubate at room temperature for 5 min., pelleted on a magnetic rack, and the bead pellet washed twice in freshly made 70% ethanol. DNA was eluted in 10 μl nuclease-free water, and each sample measured using Qubit dsDNA HS reagents. The DNA recovered was plotted as a percentage of DNA input, measured for each experiment by quantification of the ladder dilution prior to clean-up. The standard SPRI bead protocol gave the lowest total yields, followed by AmpliClean, MagBio beads and high NaCl/PEG8000 SPRI protocols. D5000 screen tape analysis showed that size selection with the standard SPRI bead protocol, and to some extent the AmpliClean beads, can account for the lower yields (see FIG. 3A). All protocols gave very consistent results, with the SPRI bead with high NaCl and high PEG 8000 concentration showing the least variability between technical replicates and independent experiments.

As shown in Table 12 and FIG. 3, SPRI beads with high NaCl/PEG 8000 concentrations perform best of all protocols tested, both in terms of total yield and variability, but all protocols give very consistent results.

TABLE 12
The means of the percentage DNA recovered for each
independent experiment (with 3 technical replicates)
is shown with the standard deviation.

| | SPRI ST | | SPRI high Na/PEG | | AmpliClean | | MagBio | |
|---|---|---|---|---|---|---|---|---|
| | Mean | S.D | Mean | S.D. | Mean | S.D. | Mean | S.D |
| Exp I | 45.3 | 4.1 | 89.7 | 3.8 | 65.1 | 12.7 | 86.4 | 9.8 |
| Exp II | 49.7 | 4 | 96.4 | 3.6 | 76.4 | 6.9 | 80 | 11.1 |
| Exp III | 61.4 | 6.5 | 97.1 | 2.7 | 78.7 | 3.1 | 91.2 | 5.3 |

### Comparison of DNA and RNA Extraction Efficiency Using Different Combinations of the Extraction (Beating) Beads and Lysis Buffers

The inventors next sought to investigate further improvement of DNA and RNA extraction methods and future separation at this stage, for later re-merging of DNA and RNA pipelines. The inventors compared the performance of multiple combinations of different types of lysis buffers and extraction beads measuring the efficiency of DNA and RNA extraction.

The inventors tested the performance of 3 types of beads (Qiagen DNeasy PowerSoil Pro Kit Power Beads, EURx Zirconia/Silica Beads, EURx Garnet sharp particles) and 6 different types of lysis buffers (Qiagen DNeasy PowerSoil Pro Kit CD1 lysis buffer, EURx Lyse T buffer, EURx GeneMATRIX Environmental DNA & RNA Purification Kit Extraction-EN buffer, EURx Gene MATRIX Soil DNA purification Kit Lyse SL buffer, EURx Universal Reagent for DNA i solation GeDI and EURx RNA Extracol) in all possible combinations:
1. Power Beads with Lyse T buffer.
2. Power Beads with CD1 buffer.
3. Zirconia/Silica Beads with Lyse T buffer.
4. Zirconia/Silica Beads with CD1 buffer.
5. Garnet sharp particles with Lyse T buffer.
6. Garnet sharp particles with CD1 buffer.
7. Power Beads with Extraction-EN buffer.
8. Power Beads with Lyse SL buffer.
9. Zirconia/Silica Beads with Extraction-EN buffer.
10. Zirconia/Silica Beads with Lyse SL buffer.
11. Garnet sharp particles with Extraction-EN buffer.
12. Garnet sharp particles with Lyse SL buffer.
13. Power beads with GeDI buffer.
14. Power Beads with Extracol buffer.
15. Zirconia/Silica Beads with GeDI buffer.
16. Zirconia/Silica Beads with Extracol buffer.
17. Garnet sharp particles with GeDI buffer.
18. Garnet sharp particles with Extracol.

20 μl aliquots of the ZymoBIOMICS Microbial Community Standard were used. 8.5 ml of foaming solution was prepared to wash the PVDF filter. The filter was cut in half after washing with scissors wiped beforehand with alcohol tissue. In total 9 filters were used. S1 protocol was followed for the extraction and purification of nucleic acids, with small modification of elution in 10 μl instead of 8. DNA and RNA concentrations were measured on Qubit and DNA quality was checked on TapeStation.

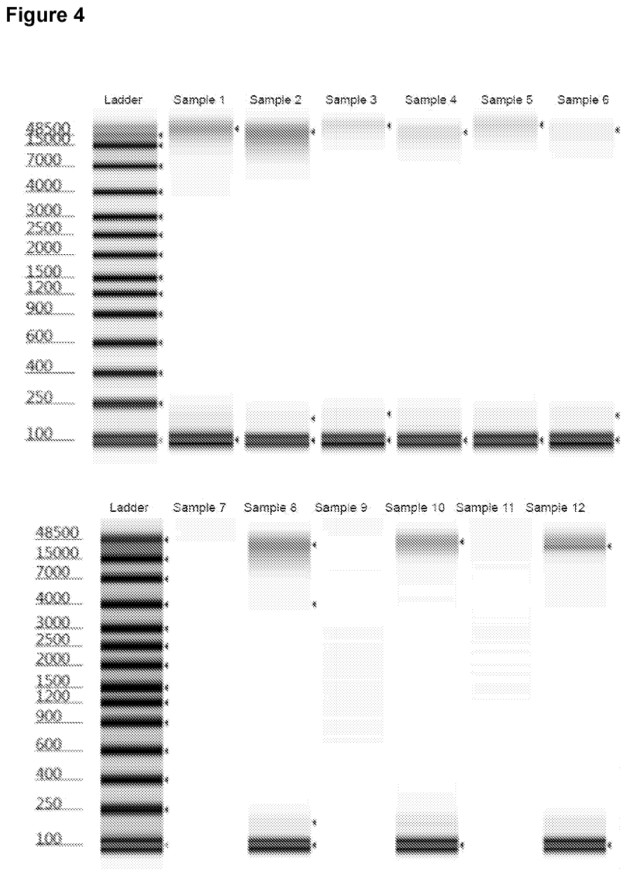

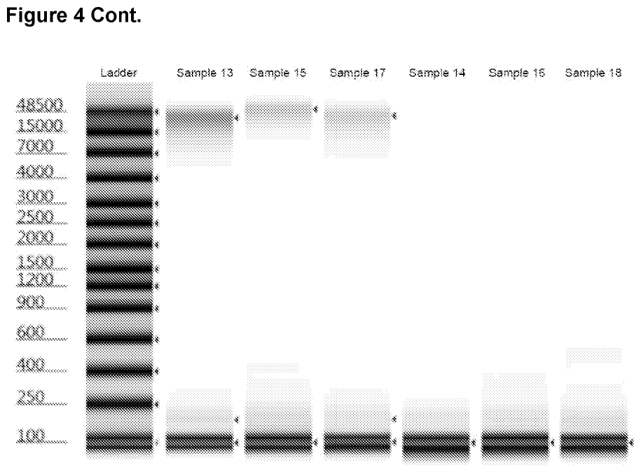

The inventors found that the highest DNA and RNA yields were extracted using an existing protocol using a combination of beads and lysis buffer coming from Qiagen DNeasy PowerSoil Pro Kit. Comparable yields of DNA were extracted using combinations of Power Beads or Zirconia/Silica beads with Lyse SL and in the case of RNA they were even higher (Table 13, FIG. 4). The cost range varies between $1.8454-$9.6082 per run.

TABLE 13
DNA and RNA concentrations and yields in particular samples measured on Qubit for
each combination of the extraction beads and lysis buffers. Highest yields are
given in bold. The sample extracted using existing protocol is Sample No 2.

| Sample | DNA concentration ng/ul | RNA concentration ng/ul | Calculated cost |
|---|---|---|---|
| No | (yield ng) | (yield ng) | per run ($) |
| 1 | 4.50 (45.0) | 14.7 (147.0) | 7.4032 |
| 2 | 7.90 (79.0) | 19.7 (197.0) | 7.1156 |
| 3 | 2.26 (22.6) | 9.54 (95.4) | 1.8454 |
| 4 | 2.94 (29.4) | 9.36 (93.6) | 8.6734 |
| 5 | 2.20 (22.0) | 5.92 (59.2) | 1.8454 |
| 6 | 1.97 (19.7) | 5.00 (50.0) | 8.6734 |
| 7 | 0 | 0 | 9.4356 |
| 8 | 7.44 (74.4) | 27.2 (272.0) | 9.6082 |
| 9 | 0 | 0 | 3.8778 |
| 10 | 4.70 (47.0) | 21.8 (218.0) | 4.0504 |
| 11 | 0 | 0 | 3.8778 |
| 12 | 4.08 (40.8) | 9.64 (96.4) | 4.0504 |
| 13. | 5.22 (52.2) | 5.54 (55.4) | 7.7148 |
| 14. | 0 | 0 | 7.4752 |
| 15. | 1.57 (15.7) | 0.920 (9.2) | 2.157 |
| 16. | 0 | 0 | 1.9174 |
| 17. | 1.96 (19.6) | 0 | 2.157 |
| 18. | 0 | 0 | 1.9174 |

Performance of Different Extraction Beads and Lysis Buffers on B. subtilis Spore Suspension

The inventors next compared the performance of the best performing combinations of different types of lysis buffers and extraction beads described above in the efficiency of DNA extraction from a very difficult material such Bacillus subtilis spore suspension for the purposes of further improvement of the extraction method.

The inventors tested the performance of 3 types of beads (Qiagen DNeasy PowerSoil Pro Kit Power Beads, EURx Zirconia/Silica Beads, EURx Garnet sharp particles) and 4 different types of lysis buffers (Qiagen DNeasy PowerSoil Pro Kit CD1 lysis buffer, EURx Lyse T buffer, EURx Universal reagent for DNA extraction Gedl and EURx Gene MATRIX Soil DNA purification Kit Lyse SL buffer) in all the following combinations:
1. Power Beads with CD1 buffer.
2. Power Beads with Lyse SL buffer.
3. Zirconia/Silica Beads with Lyse SL buffer.
4. Power Beads with Lyse T buffer.
5. Power Beads with Gedl buffer.
6. Zirconia/Silica Beads with CD1 buffer.

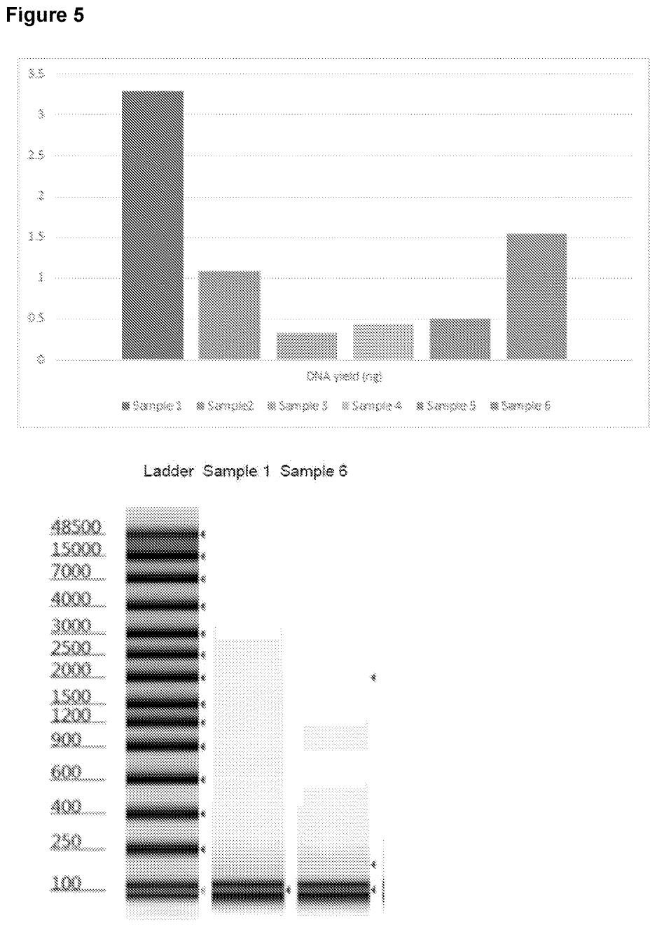

The inventor used 300 μl aliquots of B. subtilis spore suspension (Merck) as the material, which was added into the vial with the extraction beads (in case of Power beads, their amount was reduced to 0.25 g per vial). Next, 100 μl of suitable buffer was added and this mixture was ground for 5 minutes at 1,500 rpm at TissueLyser. After grinding 200 μl of Sear-Mag beads SPRI suspension was added into each vial and incubated for 5 mins at RT followed by 2× wash in 200 μl of 70% ethanol and elution in 10 μl of DNase/RNase free water for 5 mins at RT. DNA concentrations were measured on Qubit and DNA quality was checked on TapeStation (Table 14, FIG. 5).

The inventors found that the highest DNA yields were extracted using currently used combination of beads and lysis buffer coming from Qiagen DNeasy PowerSoil Pro Kit. Comparable yields of DNA were extracted using combinations of Zirconia/Silica beads from EURx with CD1 buffer suggesting that not the beads rather the lysis buffer is most important in the successful DNA extraction. The second-best performing buffer again was Lyse SL from EURx's GeneMATRIX Soil DNA Purification Kit (Table 14, FIG. 5).

TABLE 14
DNA concentrations and yields in particular samples measured
on Qubit for each combination of the extraction beads
and lysis buffers. Highest yields are given in bold.

| Sample No | DNA concentration (ng/ul) | DNA yield (ng) |
|---|---|---|
| 1 | 0.330 | 3.30 |
| 2 | 0.109 | 1.09 |
| 3 | 0.0328 | 0.33 |
| 4 | 0.0440 | 0.44 |
| 5 | 0.0500 | 0.50 |
| 6 | 0.154 | 1.54 |

### Example 2—Amplification

### RNA Amplification

polyadenylation of RNA selects for RNA molecules, ensures that all RNA can be captured by oligo d(T), ensuring specific reverse transcription of RNA, but not DNA, even with reverse transcriptases (such as Superscript IV, which is the best performing RT) that can transcribe both RNA and DNA.
Polyadenylation of RNA ensures all RNA molecules can be captured, and that the full length can be amplified and sequenced.

### DNA Amplification and Debranching

DNA from filters, were whole genome amplified (WGA), using off the shelf, Qiagen Repli-G or similar kits. Reducing volume from 100 μl->25 μl and use of homebrew beads, increased DNA concentration for subsequent WGA reactions. Enabling greater post WGA DNA concentrations.
S1 endonuclease was shown to have better debranching, increase nanopore sequencing outputs, by reducing clogging of pores. This also allowed for greater sequencing yield and multiplexing.

These optimisations are detailed further below.

### Air Sampling and End-to-End RNA Pipeline

The inventors next wanted to determine the time required for end-to-end air sampling, amplification and sequencing using the RNA pipeline.

### Air Sampling 45 Min

An air sample was collected with the Innovaprep Bobcat collector in the NHM Wildlife Garden, using continuous sampling at 200 l/min. for 45 min. (a total of 9000 l).

### Elution and Extraction 12 Min (1 Min Elution+5 Min Lysis+3 Min Ppt+1 Min Wash+2 Min Elution)

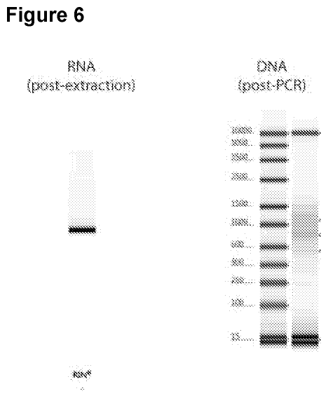

The Bobcat filter was spiked with 250 pg RNA generated from a synthetic Influenza C virus sequence (polymerase subunit PB2 construct) by pipetting the ICV-PB2 RNA directly onto the filter. The air sample was eluted from the Bobcat filter using the Bobcat foaming buffer, and filtered using a 0.1 μm PVDF membrane. The PVDF membrane was transferred to a Qiagen bead bashing tube containing 0.25 g sand and 100 μl CD1 lysis buffer, and the sample was disrupted at 25 Hz for 5 min. using Tissuelyser II. The sample was then extracted using 1×SPRI bead clean-up, with 3 min. precipitation and 2 min. elution times, and eluted in 8 μl water. Qubit RNA HS quantification showed no detectable RNA, whereas Tapestation High Sensitivity RNA screen tape analysis showed a single band at 1 kb, presumably corresponding to the ICV-PB2 RNA, see FIG. 6.

### Polyadenylation 5 Min

Polyadenylation was carried out by adding the remaining 4 μl of the sample to a 20 μl poly(A) polymerase reaction (11 μl water, 2 μl 10× buffer, 2 μl ATP, 1 μl poly(A) polymerase), and incubating at 37° C. for 5 min.

### Clean-Up 6 Min

The sample was then cleaned using 1×SPRI beads, with 3 minute precipitation and 2 minute elution time, and eluted in 5 μl water.

### Reverse Transcription 41 Min

4 μl of the cleaned sample was mixed with 1 μl ISPCR-OdTVN primer (2 μM), incubated at 70° C. for 5 min, and snap-chilled on ice. A master mix consisting of 0.8 μl water, 1.5 μl PEG 8000 (50%), 2 μl Superscript IV buffer, 0.4 μl dNTPs (100 mM), 0.2 μl ISPCR-TSO (100 UM), and 0.1 μl Superscript IV reverse transcriptase was added to the sample, and the reaction incubated at 50° C. for 30 min, and heat inactivated at 85° C. for 5 min.

### PCR 84 Min

The 10 μl RT reaction was used to set up 5 PCR reactions, each with 2 μl of the RT reaction, 4.4 μl Q5 buffer, 0.44 μl dNTPs (10 mM), 14.74 μl water, 0.2 μl ISPCR primer (10 UM) and 0.22 μl Q5 DNA polymerase. The reactions were incubated as follows: 98° C. 3 min, 25× [98° C. 20 sec, 67° C. 15 sec, 72° C. 2 min], 72° C. 5 min.

### Clean-Up 6 Min

The PCR reactions were pooled, cleaned up using 1×SPRI beads, and eluted in 6 μl water. 2 μl of the eluted sample was used for Qubit dsDNA HS quantification, see FIG. 6.

### Library Preparation 1 Min

4 μl of the sample was mixed with 8 μl of Elution Buffer and 1 μl RAP (rapid adapter mix), and loaded on a primed flow cell for sequencing.

From air collection to start of sequencing was therefore timed to 200 minutes.

Reducing Time Required for Reverse Transcription and PCR in the mcSCRB-Seq Protocol

The inventors next looked to reduce the time required for amplification in the RNA pipeline.

The 200 minute mcSCRB-seq-based RNA amplification protocol used above includes a 30 min incubation time for the reverse transcription (RT), and 2 min extension step during the PCR reaction. To determine whether it is possible to further reduce the overall time of the RNA amplification protocol, the inventors tested whether both RT time and extension times can be reduced while still producing sufficient template for sequencing (the ONT ligation sequencing kit recommends 1-200 fmol of DNA for sequencing, i.e. 60-120 ng of DNA, assuming a mean amplicon size of 1 kb). The yield of an mcSCRB-seq reaction depends on several variables, including the amount of input RNA, the incubation time of the reverse transcription reaction, and the PCR reaction conditions. Because the final yield is likely to depend on a balance between these variables, the inventors tested how RT incubation time and PCR extension times affect the total yield and amplicon size in reactions using different amounts (1 ng, 100 μg and 10 pg) of RNA inputs. The inventors have not been able to quantify the RNA present in an average air sample (9000 litres), as RNA levels in these samples are below the detection limit using the Qubit RNA HS assay. However, the inventors estimate that the RNA present in these samples is likely to be in the range of 100 pg-1 ng. The inventors have previously shown that the mcSCRBseq amplification protocol generates sufficient DNA (>300 ng) for sequencing from only 10 μg of input RNA, using 30 min RT incubation times, and 35 cycles with 2 min extension times in the PCR, and so include the 10 pg input reactions as a lower limit in these experiments.

The inventors have also used two different inputs, RNA extracted from the Zymo microbial mock community, which consists of a range of RNA sizes, as well as synthetic RNA from the PB2 subunit of Influenza C Virus (ICV). The ICV-PB2 RNA is 1 kb, and should provide a simpler assay for how RT incubation time affects cDNA lengths.

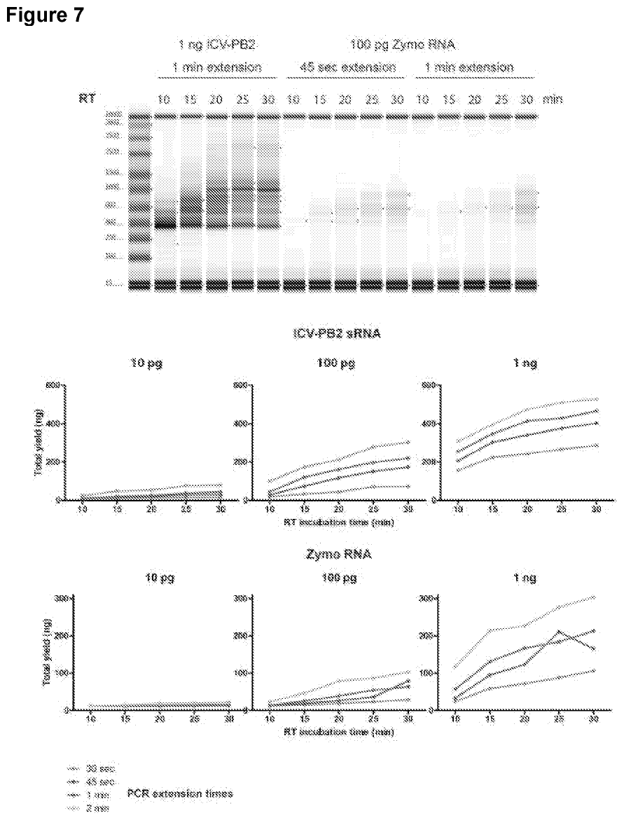

RT reactions were set up using 1 ng, 100 μg and 10 pg polyadenylated Zymo RNA and polyadenylated ICV-PB2 synthetic RNA. Briefly, 4 μl RNA was mixed with 1 μl OdTVN primer (2 μM), and incubated at 70° C. for 5 min, before snap-cooling on ice. To each reaction was added 0.8 μl water, 1.5 μl PEG 8000 (50%), 2 μl 5× Superscript IV RT buffer, 0.4 μl dNTPs (100 mM), 0.2 μl TSO (100 μM), and 0.1 μl Superscript IV Reverse Transcriptase. The RT reactions were incubated at 50° C. for 10, 15, 20, 25 or 30 min. Each RT reaction was then used to set up 4 independent PCR reactions, using 2 μl of the RT reaction mixed with 14.74 μl water, 4.4 μl Q5 buffer, 0.44 μl dNTPs (10 mM), 0.2 μl ISPCR primer (10 UM), and 0.22 μl Q5 DNA polymerase. The PCR cycling conditions used were 90° C. for 3 min, 25× [98° C. 20 sec, 67° C. 15 sec, 72° C.×min], 72° C. 5 min, 4° C. forever, where X=30 sec, 45 sec, 1 min or 2 min. PCR products were quantified using Qubit dsDNA HS, and a selection of samples were analysed using Tapestation D5000 screen tape, see FIG. 7.

Although PCR extension time would be expected to influence amplicon size, the inventors found that RT incubation time is the main determinant of amplicon length in the conditions tested here (we note that the replication rate of the Q5 DNA polymerase is 20-30 sec/kb). The expected 1 kb product from mcSCRBseq reactions using ICV-PB2 synthetic RNA as input is only detectable with RT incubation times of 20 min or more (the lower molecular weight bands in the 10 and 15 min RT reactions are likely to be short fragments resulting from internal priming of the OdTVN primer). A similar result is seen with the Zymo RNA reactions, where RT incubation times of 10 and 15 min yield amplicon sizes of approx. 400 bp and 400-600 bp respectively. With an RT incubation time of 20 or more minutes, amplicon sizes range between 400 and 1000 bp. Thus, RT incubation time can be reduced depending on the desired target length.

PCR extension time has an effect on overall yield of the PCR reactions, presumably as longer extension times increase the percentage of completed DNA fragments that subsequently act as templates in the next round of PCR. The total yield is also strongly dependent on the amount of RNA input, with 10 pg Zymo RNA input reactions showing very low yields (11-22 ng) that are similar to background (no template control and RT-control both 11 ng). However, with 30 min RT incubation time, and a PCR extension time of 2 min, 10 μg of input RNA can be amplified to yield sufficient material for sequencing, given that one RT reaction can be used to set up 5 PCR reactions, and the minimum input requirement for ligation sequencing is 60 ng. However, a typical air sample is estimated to yield RNA in the range of 0.1-1 ng. In this input range, the PCR extension time can be reduced from 2 min to 30 sec, and the RT incubation time from 30 min to 20 min (for 1 kb amplicons) or 10 min (for 400 bp amplicons), in theory reducing the overall time of the mcSCRBseq reaction by 47.5 min for 1 kb target amplicons and 57.5 min for 400 bp target amplicons. It is likely that the use of faster DNA polymerases would reduce the overall time of the mcSCRBseq protocol even further.

In summary, significant reductions in reverse transcription incubation time, as well as PCR extension time, can be achieved, depending on the desired amplicon length and the amount of starting RNA material. Assuming an air sample yields in the range of 0.1-1 ng RNA, and a target amplicon size of 400 bp (or 1 kb), reverse transcription incubation time can be reduced from 30 min to 10 min (or 20 min), and the PCR extension time from 2 min to 30 sec per cycle, resulting in a total required time of 153 min (or 143 with shorter RT step) spanning air collection to start of sequencing.

### Testing the RNA Pipeline by Making Airborne Viruses (Phages) Using a Nebuliser, Preliminary Findings

The inventors tested the detection capability of the RNA pipeline by spraying two types of phages in two different concentrations with a nebuliser while performing air sample collection.

Air sample collection: Samples were collected using the Innovaprep Bobcat at continuous setting for 45 minutes.

Phage spraying with a nebuliser: the inventors used Beurer IH55 Nebuliser (device specifications can be found here: https://lloydspharmacy.com/products/beurer-ih55-nebuliser) to spray two phages. The sprayed phages were phage Lambda and phage ¢6 at concentrations 106 and 8.33×10⁹ pfu/ml, respectively. 1 μl of Lambda phage and 1 ml of phage ¢6 was diluted in 10 ml of molecular grade water and loaded in the nebuliser reservoir. Phages were sprayed approximately 1.5 m above the ground level and at approximately 1.5 m distance from the Bobcat air sampler by circulating around Bobcat. The nebulising process lasted about 10 minutes, but the air sample collection was continued as normally.

Sample initial processing: Elution from the Bobcat filter was done using supplied foaming buffer and capture on 0.1 μm PVDF membrane. Samples were stored at −80° C. until further processing. RNA extraction: Sample (PVDF filter) was transferred to a PowerBead Pro Tube, using a reduced amount of beads (0.25 g), then 100 μl of CD1 buffer (DNeasy PowerSoil Pro Kit) was added. Sample was ground for 5 minutes at 1500 rpm (=25/s) in a TissueLyser II. The sample was spun in a microcentrifuge for 1 minute at maximum speed and the supernatant transferred to a 1.5 ml tube. A 1×SPRI bead clean-up was performed (100 μl SPRI beads, followed by 2× wash in 200 μl 70% EtOH) and eluted in 8 μl of DNase/RNase free water for 5 minutes. RNA extraction was quantified using Qubit RNA HS reagents, which yielded 76.16 ng of RNA (concentration: 9.52 ng/μl).

### Polyadenylation Tailing:

1) 4 μl of the sample was used in a 20 μl of poly(A) polymerase reaction (11 μl water, 2 μl 10× buffer, 2 μl ATP, 1 μl poly(A) polymerase), and incubated at 37° C. for 5 min.
2) A 1×SPRI bead clean-up was performed, with 3 minute precipitation and 2 min elution time, and the polyadenylated sample was eluted in 5 μl water.

### Reverse Transcription:

1) 4 μl of the cleaned sample was mixed with 1 μl ISPCR-OdTVN primer (2 μM), incubated at 70° C. for 5 min, and snap-chilled on ice.
2) To the RNA-primer mix was added a master mix consisting of 0.8 μl water, 1.5 μl PEG 8000 (50%), 2 μl Superscript IV buffer, 0.4 μl dNTPs (100 mM), 0.2 μl ISPCR-TSO (100 UM), and 0.1 μl Superscript IV reverse transcriptase to the sample.
3) Incubate the reaction at 50° C. for 30 min, and heat inactivate at 85° C. for 5 min.

### PCR:

1) The 10 μl RT reaction was used to set up 5 PCR reactions, each with 2 μl of the RT reaction, 4.4 μl Q5 buffer, 0.44 μl dNTPs (10 mM), 14.74 μl water, 0.2 μl ISPCR primer (10 UM) and 0.22 μl Q5 DNA polymerase.
2) The reactions were cycled as follows: 98° C. 3 min, 25× [98° C. 20 sec, 67° C. 15 sec, 72° C. 2 min], 72° C. 5 min. 3) The PCR reactions were pooled, and cleaned up using 1×SPRI beads, and eluted in 6 μl water. 2 μl of the eluted sample was used for Qubit dsDNA HS quantification, and 1 μl for the TapeStation analysis. The remaining sample was used to prepare a sequencing library, using the ONT Ligation Sequencing Kit.

Bioinformatic analysis: 400,000 reads (approx. 5% of the total number of reads obtained) were classified via the Marti pipeline (megaBLAST-NTL/LCAParse). The majority of reads mapped to plants (79.2%), fungi (3.2%) and bacteria (1.4%), while a total of 10 reads mapped to Pseudomonas virus phi6 (0.0025%). No lambda phage reads were recovered, suggesting that the total amount of lambda phage loaded in the nebuliser may have been too low to detect.

The inventors have shown that the existing protocol for the RNA pipeline can detect air-microbiome elements. The post-extraction Qubit RNA concentration measure (76.16 ng of total RNA yield) in the sample already indicated successful detection of the phage @6, which was confirmed by the sequencing results analysis.

### WGA MaxVolume Method

The inventors diluted Zymo Mock DNA to a concentration of 10 ng/μl in water and prepared the following WGA reactions:
(1) Control reaction: 1 μl DNA, 1 μl DLB denaturation solution (diluted), 2 μl Stop solution (diluted), 15 μl RepliG buffer, 1 μl RepliG Ultrafast polymerase
(2) WGA MaxVolume reaction: 3.675 μl DNA, 0.125 μl DLB denaturation solution (undiluted), 0.20 μl Stop solution (undiluted), 15 μl RepliG buffer, 1 μl RepliG Ultrafast polymerase

The inventors incubated the reactions for 90 minutes at 30° C. with 3 minutes final enzymatic heat kill step at 65° C. Both reactions were quantified using Qubit 2.0 Broad Range reagents (pre-cleanup).

The inventors observed that by using neat denaturation and stop solutions higher DNA inputs in a WGA reaction are achieved leading to achieving higher WGA yields WGA (Table 15).

TABLE 15
Qubit 2.0 Broad Range quantification of a standard
and MaxVolume RepliG Ultrafast WGA reaction.

| Reaction | Qubit 2.0 BR [ng/μl] | Ng total |
|---|---|---|
| Standard | 59.4 | 1188 |
| MaxVolume | 156 | 3120 |

On bead WGAThe EquiPhi WGA kit uses heat to denature DNA ahead of the amplification step and also works in a larger reaction volume than the Repli-G system tested before. An on-bead WGA step would reduce processing time (bead elution and liquid handling time) and could increase yield by maximising inputs.

The inventors compared WGA DNA yields from a standard bead beat to WGA via DNA elution into water versus bead beat to DNA elution in the WGA primers at 95° C. Zymo mock cells were bead beaten in 100 μl lysis buffer and 250 mgs beads in a SuperFast-Prep2 for 30 s. The tubes were spun down, and the supernatant transferred into a fresh 1.5 ml lobind tube. A 1× bead clean-up was undertaken with 2×70% EtOH washes.

For the control the DNA was eluted from the beads with a 2-minute incubation in 5 μl water at room temperature. The DNA was then transferred to a new tube and combined with 5 μl of water and 2 μl of exo resistant random hexamers and heated to 95° C. for 3 minutes.

For the on bead test the DNA was eluted from the beads directly into 10 μl of water combined with 2 μl of exo resistant random hexamers and heated to 95° C. for 3 minutes (i.e. no 2 minute elution step).

Both tubes had 2 μl dNTPs, 0.2 μl DTT, 1.8 μl H2O, 2 μl Buffer and 2 μl Enzyme added and were then heated to 45° C. for 30 m. Post incubation 30 μl of water was added and a 1× Bead clean-up performed with amplified DNA eluted in 20 μl water.

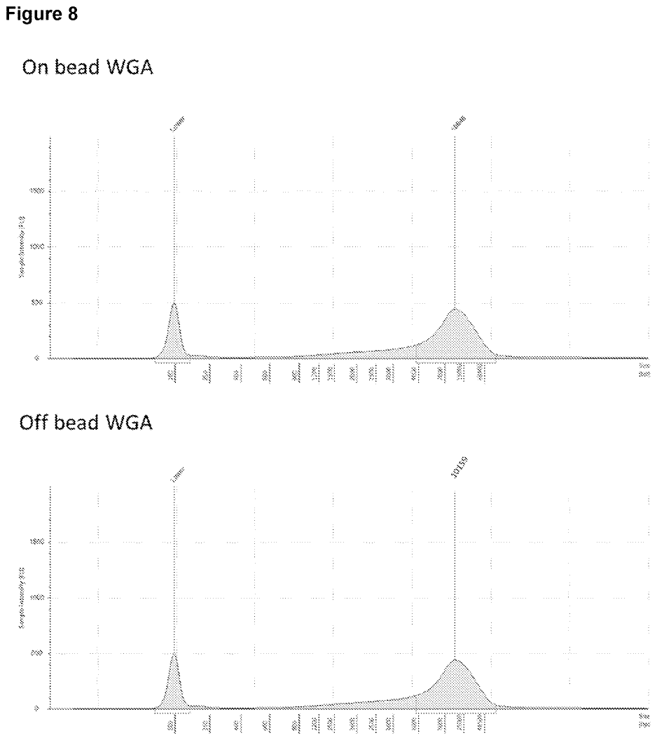

Although yield was slightly lower (around 80% of the off-bead WGA) there was still more than sufficient material generated for sequencing (Table 16). When we compared the WGA material on the Agilent Tapestation Genomic Tape the inventors observed very little difference between the electropherograms suggesting that the beads had not inhibited the WGA (FIG. 8). Results also suggested that the inventors might be able to further optimise the amplification incubation saving even more time e.g. shorter WGA incubation times. Fragment sizes of the amplified material are very similar indicating no negative effect on downstream read lengths.

TABLE 16
DNA concentrations achieved with
control WGA versus on-bead WGA

| | DNA concentration (ng/ul) |
|---|---|
| Control WGA | 31.4 |
| On-bead WGA | 26.2 |

### Example 3—Sequencing

The present inventors have developed optimised sequencing methods to allow increased sensitivity to extremely low abundant organisms and achieve a significant reduction in time to sequencing, from 180 min before the project start to 60 min.
Showed that we can detect sample present down to at least 1 in 200 000;
Reduced the amount of starting DNA, from 400 ng to 5 ng DNA in full and half reaction volumes. With increased collection times, can get sufficient material in 10 m collection to not need WGA;
Demonstrated the ability to reduce the loading amount of DNA resulting in removing the need for amplification;
Added a step for nuclease flush to re-load DNA which enables flowcell reuse up to 8 times. Flow cells are expensive as such this improves cost effectiveness of the sequencing process.

These optimisations are detailed further below.

Comparison of Time from Sample Collection to Sequencing in Two Pipelines

To test the current pipeline the inventors assumed an air collection time of 1 minute 30 seconds and then timed how long it took to process samples following pipeline 1 and the pipeline 2.

For comparison purposes both pipelines were started with the same input material. Zymo mock cells were bead beaten in a SuperFast-Prep2 for 30 s at Speed code 20 and the supernatant recovered. A 1× home brew bead clean-up was performed precipitating DNA for 3 minutes, washing twice with 70% ethanol and then eluting in 10 μl DNase free water. DNA was then normalised to 1 ng/μl and then 3.75 μl used as input for either pipelines 1 or 2.

### Pipeline 1

WGA was performed using the Qiagen Repli-G kit. To 3.75 μl of DNA 0.25 μl of neat DLB was added, mixed and then incubated for 3 minutes at room temperature. The 0.4 μl of stop solution and mixed followed by 16 μl of polymerase buffer and 1 μl of enzyme. These were then thoroughly mixed, spun and then incubated for 90 minutes at 80 C followed by 80 C for 3 minutes. A 1× home brew bead clean-up was performed precipitating DNA for 3 minutes, washing twice with 70% ethanol and then eluting in 10 μl DNase free water. An S1 nuclease treatment was performed by combing 500 ng of WGA DNA with 4 μl of buffer and 1 μl of enzyme and making the volume up to 20 μl of water. This was then thoroughly mixed, spun and then incubated for 10 minutes at 37 C. A 1× home brew bead clean-up was performed precipitating DNA for 3 minutes, washing twice with 70% ethanol and then eluting in 10 μl DNase free water.

### Pipeline 2

WGA was performed using the Thermo-Fisher EquiPhi kit. To 3.75 ng of DNA in 10 μl of water 2 μl of random hexamers were added then thoroughly mixed, spun and then incubated for 3 minutes at 95 C and then cooled on ice. To this 2 μl of 10 mM dNTPs, 1.8 μl of water, 0.2 μl of 100 mM DTT, 2 μl of buffer and 2 μl of enzyme were added then thoroughly mixed, spun and then incubated for 30 minutes at 45 C followed by 80 C for 3 minutes. A 1× home brew bead clean-up was performed precipitating DNA for 3 minutes, washing twice with 70% ethanol and then eluting in 10 μl DNase free water.

An S1 nuclease treatment was performed by combing 500 ng of WGA DNA with 4 μl of buffer and 1 μl of enzyme and making the volume up to 20 μl of water. This was then thoroughly mixed, spun and then incubated for 10 minutes at 37 C. A 1× home brew bead clean-up was performed precipitating DNA for 3 minutes, washing twice with 70% ethanol and then eluting in 10 μl DNase free water.

The DNA yields are shown below in Table 17.

TABLE 17
DNA yields for pipelines 1 and 2

| | Post WGA (ng) | Post S1 (ng) |
|---|---|---|
| Task B | 1575 | 430 |
| Task C | 579 | 330 |

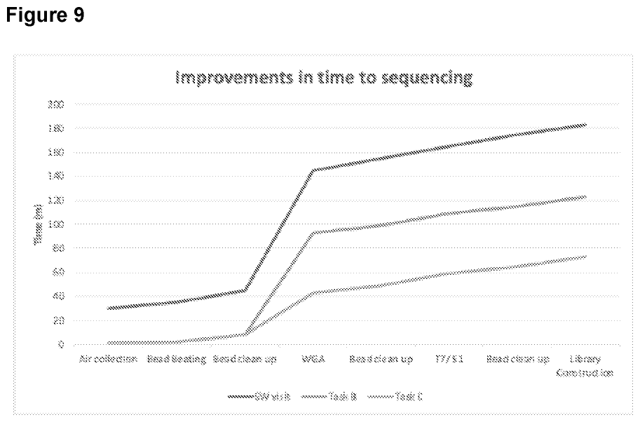

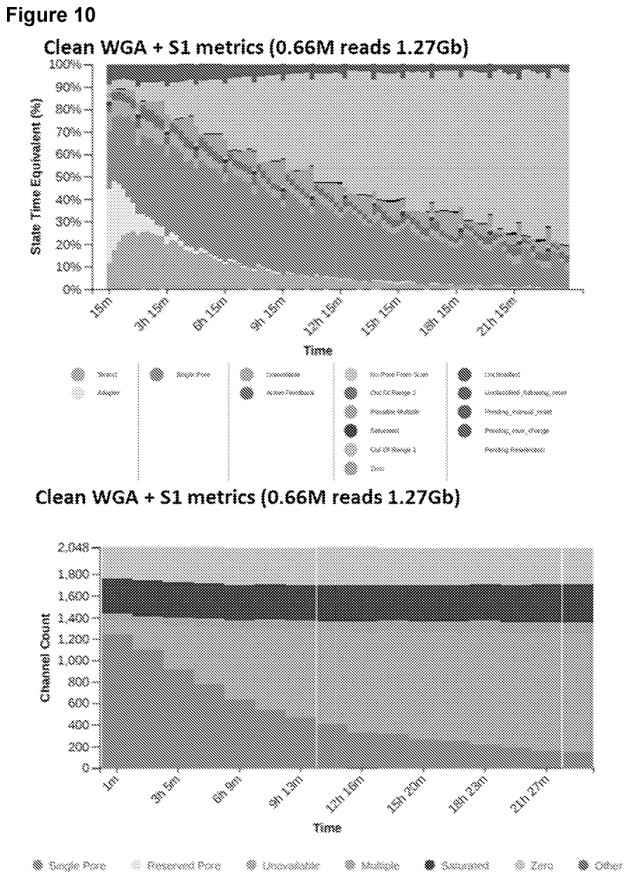

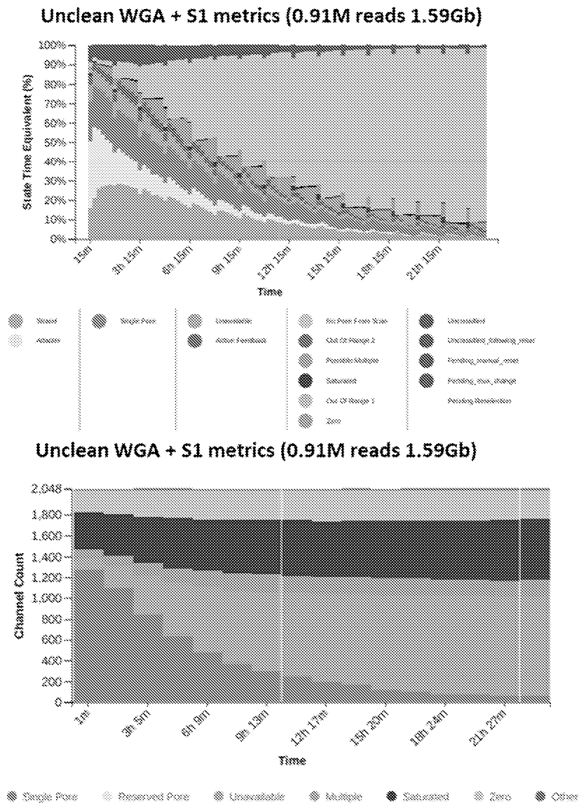

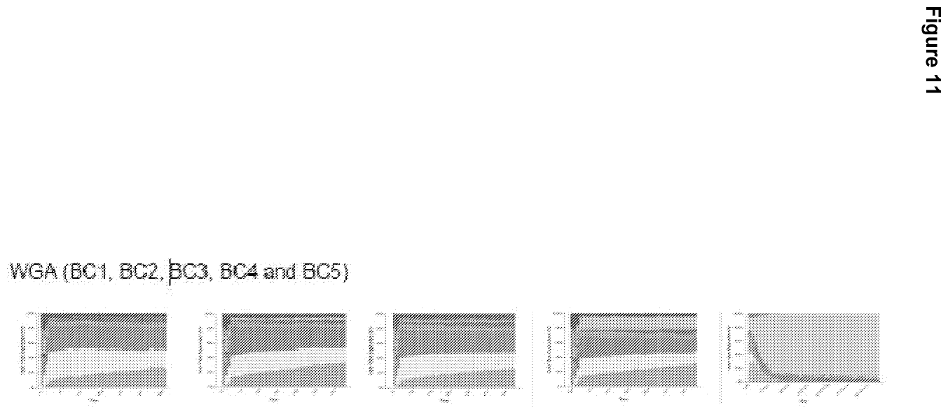

The cumulative times for each pipeline are shown in FIG. 9. The results show significant improvements such that samples can be processed within one hour. With the optimisation steps achieved in pipeline 1 combined with those made in pipeline 2, time savings have been achieved with air collection, bead beating, bead clean ups and WGA. The total time from beginning air collection to being ready to sequence is now <75 minutes.

WGA Debranching with: S1, T7, Bst, gTube

We prepared two max volume WGA reactions using 10 ng Zymo Mock DNA input which we amplified for 90 minutes at 30° C. with a final heat denaturation step for 3 minutes at 65° C. We pooled both reactions to a pool with 40 μl volume. We measured the concentration of the pool using Qubit 2.0 Broad Range reagents (184 ng/μl) and cleaned 20 μl of the pool with a 1×SPRI bead cleanup. We quantified the cleaned reaction using Qubit 2.0 Broad Range regents (132 ng/μl) and proceeded with the following reactions:
1) WGA clean+S1: 15 μl clean WGA, 4 μl Thermo S1 Nuclease buffer 5×, 1 μl S1 nuclease for 10 minutes at 37° C.
2) WGA unclean+S1: 20 μl uncleaned WGA, 8 μl Thermo S1 Nuclease buffer 5×, 1 μl S1 nuclease, 11 μl water, for 10 minutes at 37° C.

Note: The uncleaned WGA reaction was diluted and performed in 40 μl total volume to better adjust the S1 nuclease reaction buffer conditions: 200 mM NaOAc (pH 4.5 at 25° C.), 1.5M NaCl, 20 mM ZnSO 4 (5× buffer), which differ from the phi29 reaction buffer; e.g.: 330 mM Tris-acetate (pH 7.9 at 37° C.), 100 mM magnesium acetate, 660 mM potassium acetate, 1% Tween 20, 10 mM DTT (10× buffer). We kept the enzyme concentration the same, as higher S1 nuclease concentrations can lead to increased dsDNA degradation according to the supplier guidelines (see: https://www.thermofisher.com/order/catalog/product/EN0321 #/EN0321).

After incubation we immediately proceeded with a 1×SPRI bead cleanup, eluted in 10 μl 1× TE buffer and quantified both reactions using Qubit 2.0 Broad Range reagents: (1) WGA clean+S1 [184 ng/μl], (2) WGA unclean+S1 [132 ng/μl].

The inventors prepared 400 ng of each reaction in 7.50 μl, diluted with water and proceeded with a standard protocol SQK-RAD004 library preparation for sequencing on a full ONT flowcell. Each reaction was sequenced for 24 hours, after 24 hours we analysed the sequencing reports:

TABLE 18
S1 nuclease digest sequencing performance.

| | | | | | | | |
|---|---|---|---|---|---|---|---|
| | | Reads | Bases | | #reads | MB | |
| | Channels | generated | generated | N50 | generated/ | generate/ | Run |
| Experiment | start | (K) | (Mb) | (Kb) | channel | channel | length |

S1 nuclease test

| | | | | | | | |
|---|---|---|---|---|---|---|---|
| WGA clean + | 1236 | 661.5 | 1270 | 3.08 | 535 | 1.03 | 24 h |
| S1 | | | | | | | |
| WGA | 1273 | 917.15 | 1590 | 2.91 | 720 | 1.25 | 24 h |
| unclean + | | | | | | | |
| S1 | | | | | | | |

Previous WGA only Sequencing

| | | | | | | | |
|---|---|---|---|---|---|---|---|
| WGA | 1397 | 303.33 | 646.61 | 3.30 | 217 | 0.46 | 21 h 15 m |

Although the ONT flowcell loaded with the S1 nuclease digest applied on the non-cleaned WGA reaction produced higher yields to the control reaction (S1 nuclease applied on clean WGA product) we observed higher pore ‘dropout’ rates when applying S1 nuclease on unclean WGA reactions. Therefore, if saving time is key the clean-up step between WGA and S1 digest can be skipped, if pore activity health is key (e.g. for multiplexing, etc.) the clean-up step should be performed.

### WGA v T7 v S1 Flowcell Reuse

Either S1 or T7 treatment adds around 20 m to the protocol. If nuclease flush on the nanopore flowcell of WGA only library (i.e. no debranching) can remove molecules that are blocking pores and free them for sequencing the next library (sample) then this could circumnavigate the need for any debranching.

WGA material generated using 20 ng input into WGA and 60 m extension at 30° C. Material then treated with either S1 or T7. 50 ng then used in half reaction volume RBK004 rapid library and loaded onto a flowcell. Time taken to 50 000 passed reads noted, a nuclear flush undertaken and then the flowcell loaded with a new library with a different barcode.

The results are shown in Table 19 below:

TABLE 19

| | | Pores available | Run Time to 50000 | From start of | |
|---|---|---|---|---|---|
| | | for sequencing at | passed filter reads | sequencing | Read N50 |
| Library | Barcode | start of run | (m) | (m) | (kbp) |
| WGA | 1 | 509 | 51 | 47 | 3.11 |
| | 2 | 500 | 44 | 40 | 3.12 |
| | 3 | 496 | 55 | 51 | 3.36 |
| | 4 | 423 | 43 | 39 | 2.88 |
| | 5 | 405 | 51 | 47 | 3.23 |
| WGA × S1 | 1 | 512 | 73 | 69 | 3.56 |
| | 2 | 508 | 45 | 41 | 3.59 |
| | 3 | 504 | 54 | 50 | 3.69 |
| | 4 | 502 | 47 | 43 | 3.49 |
| | 5 | 449 | 46 | 42 | 3.27 |
| NGA × T7 | 1 | 503 | 47 | 43 | 2.27 |
| | 2 | 496 | 40 | 36 | 2.01 |
| | 3 | 481 | 57 | 53 | 2.23 |
| | 4 | 461 | 36 | 32 | 1.75 |
| | 5 | 451 | 44 | 40 | 2.14 |

Average time to 50 000 passed filter reads across all 5 loadings were 44.8 m for WGA, 44 m for WGA×S1 and 40.8 m for WGA×T7. For the last loading the flowcells were left to run and as expected in the WGA only run the pores blocked quickly and only 475 k reads were generated. The benefit of the debranching was highlighted as the S1 treated sample generated >1 million passed filter reads and the T7>1.8 million. Again the greater number observed in the T7 would have been, in part, down to the smaller read N50.

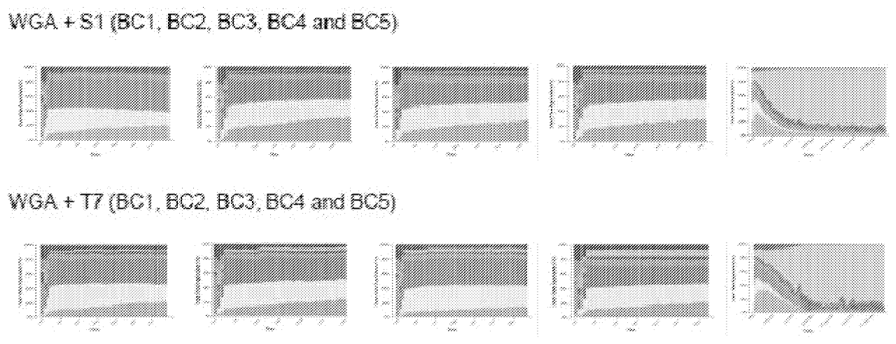

Mux scans of the various runs are shown in FIG. 11. There was little difference between WGA, WGA+S1 and WGA+T7 for the first 4 four barcodes where runs were limited to only 50 k passing filter reads. However, for the last run there is a more noticeable drop off in available pores but this is for an extended run.

Although the inventors observed that the T7 treated WGA library was consistently quicker to 50000 passed filter reads this was down to the lower read N50 so probably as a result of slightly less DNA added. Overall, the flow cells stood up well to nuclease flushing and through 4 washes the time to 50 000 passed filter reads was consistent. This suggests that the inventors might not need to debranch to reuse flowcells as the nuclease flush treatment is sufficient to unblock pores.

### Example 4—Comparing Protocols Performance for DNA Extraction, Amplification and Debranching

The inventors compared the performance of two protocols used so far for DNA extraction, amplification and debranching to determine best performing protocol time wise and yield wise for future automatization testing. Because the two protocols are using different enzymes for debranching, old one uses T7 endonuclease, new one S1 endonuclease, we therefore named them as T7 protocol and S1 protocol, respectively.

In order to compare the two protocols, the ZymoBIOMICS Microbial Community Standard was used. 8.5 ml of foaming solution was prepared to wash the PVDF filter. The filter was cut in half after washing with scissors wiped beforehand with alcohol tissue. One half was treated with T7 protocol and the second half with S1 protocol.

The inventors found that S1 protocol gives slightly higher DNA yield after the final step of debranching, which was checked on Qubit (Table 20). The DNA quality between two protocols was comparable, which was checked on TapeStation. Regarding time, the S1 protocol also saves time of 12 minutes at the stage of debranching.

TABLE 20
DNA yield in subsequent steps measured on Qubit and on
TapeStation (only the final stage with this method).

| | DNA extraction | Amplification stage | Debranching stage | |
|---|---|---|---|---|
| Protocol used | stage (Qubit) | (Qubit) | Qubit | TapeStation |
| T7 | 12.8 ng/μl | 101.00 ng/μl | 127.00 ng/μl | 176.00 ng/μl |
| S1 | 12.1 ng/μl | 146.00 ng/μl | 166.00 ng/μl | 253.00 ng/μl |

1. A method for extracting a nucleic acid from a sample comprising one or more cells comprising nucleic acid, the method comprising:
providing a sample comprising one or more cells;
lysing the one or more cells by contacting the sample with a solution comprising a chaotropic agent;
mechanically disrupting the sample comprising one or more cells; and
contacting the sample with magnetic particles;
wherein the method comprises contacting the sample with a non-RNA carrier.

2. The method according to claim 1, wherein the non-RNA carrier is selected from a synthetic polymer, DNA, synthetic nucleic acid, or a polysaccharide.

3. The method according to claim 1, wherein the chaotropic agent is selected from phenol, ethanol, guanidinium, urea, iodide and lithium perchlorate.

4. The method according to claim 1, wherein the solution comprising the chaotropic agent is used at a volume between 50 μl to 250 μl.

5. The method according to claim 1 wherein the sample is contacted with between 100 mg to 350 mg of the magnetic particles.

6. The method according to claim 1, wherein the method further comprises a step of amplifying the nucleic acid to produce an amplified nucleic acid sample, optionally wherein the amplification step comprises amplifying DNA via whole genome amplification, to produce a sample of amplified DNA.

7. (canceled)

8. The method according to claim 6, wherein the method comprises a step of debranching the amplified DNA, comprising contacting the amplified DNA with an endonuclease selected from SI, T7.

9. The method according to claim 6, wherein the amplification step comprises amplifying RNA via a method selected from RT-PCR, isothermal amplification or rolling circle amplification, to produce a sample of amplified RNA.

10. The method according to claim 6, wherein the amplification step comprises a step of polyadenylating RNA, optionally wherein the amplification step further comprises reverse transcription using an oligonucleotide deoxythymidine homopolymer primer.

11. (canceled)

12. The method according to claim 6, wherein the amplification step comprises contacting the RNA with a reverse transcriptase preferably Superscript IV.

13. The method according to claim 6, wherein the amplification step comprises amplifying the RNA with primers that attach a CLICK chemistry active group to the amplified cDNA, optionally wherein the CLICK chemistry active group is selected from a dibenzocyclooctyne group, or an azide group.

14. (canceled)

15. The method according to claim 1, wherein the method further comprises a step of sequencing the nucleic acid, optionally wherein the sequencing comprises whole genome sequencing, whole exome sequencing, targeted sequencing or metagenomic sequencing.

16. (canceled)

17. The method according to claim 15 wherein the sequencing produces sequencing reads of >1 kb.

18. The method according to claim 1, wherein the sample is a sample obtained from the environment.

19. The method according to claim 1, wherein the sample is an air, water, soil sample, optionally wherein the air sample is collected via Coriolis micro, FLIR IBAC 2, Innovaprep ACD-200 Bobcat.

20. (canceled)

21. The method according to claim 1, wherein the sample is filtered prior to performing the method of claim 1.

22. The method according to claim 1, wherein the step of lysing one or more cells minimises nucleic acid fragmentation.

23. A method for identifying pathogens and/or allergens present in an environmental sample, the method comprising
obtaining a sample from the environment comprising one or more cells;
extracting nucleic acid from said sample by lysing the one or more cells by contacting the sample with a solution comprising guanidinium; mechanically disrupting the sample comprising one or more cells; contacting the sample with magnetic particles; and contacting the sample with a non-RNA carrier molecule;
amplifying the nucleic acid; and
sequencing the nucleic acid.

24. A method for organism profiling of an environmental sample, the method comprising
obtaining a sample from the environment comprising one or more cells;
extracting nucleic acid from said sample by lysing the one or more cells by contacting the sample with a solution comprising a chaotropic agent; mechanically disrupting the sample comprising one or more cells; contacting the sample with magnetic particles; contacting the sample with a non-RNA carrier molecule;
amplifying the nucleic acid; and
sequencing the nucleic acid and identifying the microorganisms present in the environmental sample.

25. A kit for the extraction of nucleic acid, comprising a solution comprising a chaotropic agent, magnetic particles, a non-RNA carrier and optionally instructions for use.
